(19) United States
(12) Patent Application Publication
(10) Pub. No.: US 2025/0102500 A1
(43) Pub. Date: Mar. 27, 2025

(54) PROCESS FOR HIGH PERFORMANCE BARCODED MAGNETIC BEADS

(71) Applicant: APPLIED BIOCODE INC., Santa Fe Springs, CA (US)

(72) Inventors: Chung-Jen Hou, Pensacola, FL (US); Gao Chen, Hawthorne, CA (US)

(21) Appl. No.: 18/471,679

(22) Filed: Sep. 21, 2023

(51) Int. Cl. G01N 33/543; C08G 59/14; C12Q 1/6837

(52) U.S. Cl. CPC G01N 33/5434; C08G 59/1477; C12Q 1/6837; G01N 2333/90203

(57) ABSTRACT

The present disclosure relates to a new process for construction of controlled structures on the surface of modified digital barcoded magnetic beads (BMB) with higher capacity, higher fluorescence intensity, wider dynamic range, and improved sensitivity in clinical applications.

### BACKGROUND

Photo-curable epoxy compositions containing EPON SU-8 resin, EPON 1002F, or other bi-functional or multifunctional epoxy resins may be used to cast films or fabricate beads, magnetic beads, or magnetic beads. The resulting various kinds of films, micro beads, magnetic beads, or magnetic beads containing nickel barcodes may find use in clinical or biological applications. The invention relates generally to assay beads and methods for use thereof to carry out multiplexed bioassays with digital magnetic beads, and more particularly multiplexed bioassays using micro-volume samples, such as protein and nucleic acid analysis.

There are two major issues in clinical tests. One is how to increase sensitivity of tests, i.e., better signal/noise ratio, to avoid false positive or false negative test results. The other is how to increase the dynamic range of tests.

There is accordingly a need for epoxy resin-based platforms with increased sensitivity, reduced false negatives, and improved dynamic range for clinical and biological applications.

### SUMMARY

Provided herein are biological assay devices comprising a reactive solid substrate linked to one or more amino-spacerⁿ-n Carboxyl (COOH) moieties, said amino-spacerⁿ-n Carboxyl (COOH) moieties having a first end comprising a terminal amino group linked to the solid substrate, and a second end comprising n terminal carboxylic acid functionalities linked to one or more biological probe molecules; wherein each of the one or more amino-spacerⁿ-n Carboxyl (COOH) moieties comprises two or more branches each independently comprising one or more nitrogen atoms and a chain comprising a linear C₁₋₂₀ alkylene, C₁₋₆ alkylene oxide, or polyalkylene oxide; and n is an integer from 2 to 6. Further provided are amino-spacerⁿ-n Carboxyl (COOH) moieties having a first end comprising a terminal amino group linked to the solid substrate, and a second end comprising n terminal carboxylic acid functionalities linked to one or more biological probe molecules; wherein each of the one or more amino-spacerⁿ-n Carboxyl (COOH) moieties comprises two or more branches each independently comprising one or more nitrogen atoms and a chain comprising a linear C₁₋₂₀ alkylene, C₁₋₆ alkylene oxide, or polyalkylene oxide; and n is an integer from 2 to 6. In some embodiments, the amino-spacerⁿ-n Carboxyl (COOH) moieties are selected from Compound 1, Compound 2, Compound 3, Compound 4, Compound 5, and Compound 6.

Particularly provided are biological assay devices comprising an epoxy-containing SU-8 barcoded magnetic bead substrate linked to one or more amino-spacerⁿ-n Carboxyl (COOH) moieties described herein.

Also provided are methods of detecting a substance in a biological sample, comprising contacting the biological sample with the biological assay devices described herein.

Further provided are methods of preparing the amino-spacerⁿ-n Carboxyl (COOH) moieties described herein. Further provided are methods of preparing the biological assay devices comprising reactive solid substrate linked to one or more amino-spacerⁿ-n Carboxyl (COOH) moieties described herein.

### BRIEF DESCRIPTION OF THE DRAWINGS

FIG. 1 shows the structure of Compound 1 disclosed herein (Amino-Spacer¹-1 C).

FIG. 2 shows the structure of Compound 2 disclosed herein (Amino-Spacer²-2 C).

FIG. 3 shows the structure of Compound 3 disclosed herein (Amino-Spacer³-3 C).

FIG. 4 shows the structure of Compound 4 disclosed herein (Amino-Spacer⁴-4 C).

FIG. 5 shows the structure of Compound 5 disclosed herein (Amino-Spacer⁵-5 C).

FIG. 6 shows the structure of Compound 6 disclosed herein (Amino-Spacer⁶-6 C).

### DETAILED DESCRIPTION

One way to solve the two major issues in clinical tests is to increase the molecular probe density on the surface of modified digital barcoded magnetic beads (BMB). There are several approaches to increase probe density on modified BMB surface by optimizing process conditions. During surface modification process of BMB, the higher concentration of reagent or reactive oligomer/polymer will increase the reactive binding sites for probes on the resulting modified BMB surface.

Each reactive binding site on the surface of BMB can be further amplified by surface modifying reagents with controlled structure as described in this invention to increase surface functionalities and reduce non-specificity for molecular probes.

A micro bead having a digitally coded structure is partially transmissive and opaque to light. The pattern of transmitted light is determined by the design to decode the bead. The coded bead may be structured into a series of alternating light transmissive and opaque sections, with relative positions, widths and spacing resembling a 1D or 2D bar code image. To decode the image, the alternating transmissive and opaque sections of the body are scanned in analogous fashion to bar code scanning. The coded bead may be coated or immobilized with a capture or probe to affect a desired bioassay. The coded bead may include a paramagnetic material. The barcode microbeads or micro pallets are typically fabricated by photolithography. Thousands or millions of micro beads or micro patterns can be fabricated on a micro slide, a glass or a silicon wafer. Although photopolymers are commonly used in the semiconductor industry, however, all the semiconductor industry photopolymers are not biocompatible, which means that it is very difficult to immobilize biomolecules, such as proteins, oligonucleotides or cells, on the surface of these materials, especially long-term stability is required for storage.

Barcoded magnetic beads made from SU-8 photoresist contain reactive epoxy functionalities on bead surface. The reactive bi-functional or multifunctional compounds, oligomer, prepolymer, resins, or functional polymer containing amino functionalities can react with surface epoxy groups.

Provided herein are biological assay devices comprising a reactive solid substrate linked to one or more amino-spacerⁿ-n Carboxyl (COOH) moieties, said amino-spacerⁿ-n Carboxyl (COOH) moieties having a first end comprising a terminal amino group linked to the solid substrate, and a second end comprising n terminal carboxylic acid functionalities linked to one or more biological probe molecules;

wherein each of the one or more amino-spacerⁿ-n Carboxyl (COOH) moieties comprises two or more branches each independently comprising one or more nitrogen atoms and a chain comprising a linear C₁₋₂₀ alkylene, C₁₋₆ alkylene oxide, or polyalkylene oxide; and n is an integer from 2 to 6.

In some embodiments, the one or more amino-spacerⁿ-n Carboxyl (COOH) moieties comprise two or more branches derived from combinations of initial amino-spaceⁱ-i C (COOH), epoxy-spacer^{m}-m C (COOH), bifunctional epoxy compounds, and multifunctional amines, wherein i and m are each independently integers from 1 to 20.

In some embodiments, the one or more amino-spacerⁿ-n Carboxyl (COOH) moieties have a structure selected from amino-spacer²-2 C (COOH), amino-spacer³-3 C (COOH), amino-spacer^{4-I}-4 C (COOH) [1 C+3 C], amino-spacer^{4-II}-4 C (COOH) [2 C+2 C], amino-spacer^{5-I}-5 C (COOH) [2 C+3 C], amino-spacer^{5-II}-5 C (COOH) [1 C+4 C (1 C+3 C)], amino-spacer^{5-II}-5 C (COOH) [1 C+4 C (2 C+2 C)], and amino-spacer^{6-(c)}-6 C (COOH) [3 C+3 C]:

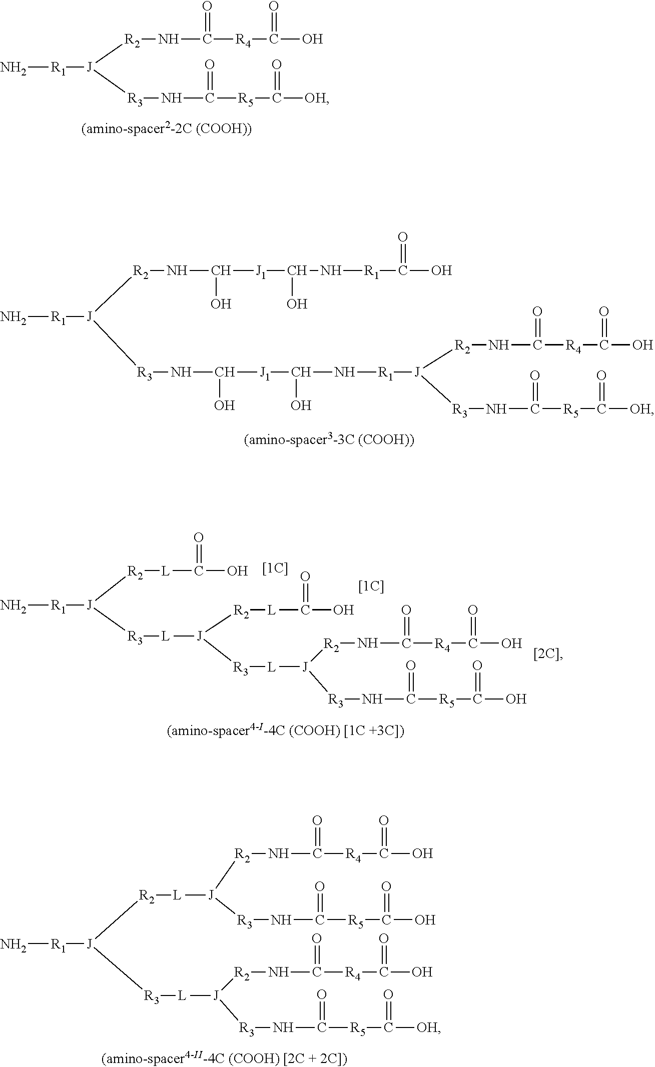

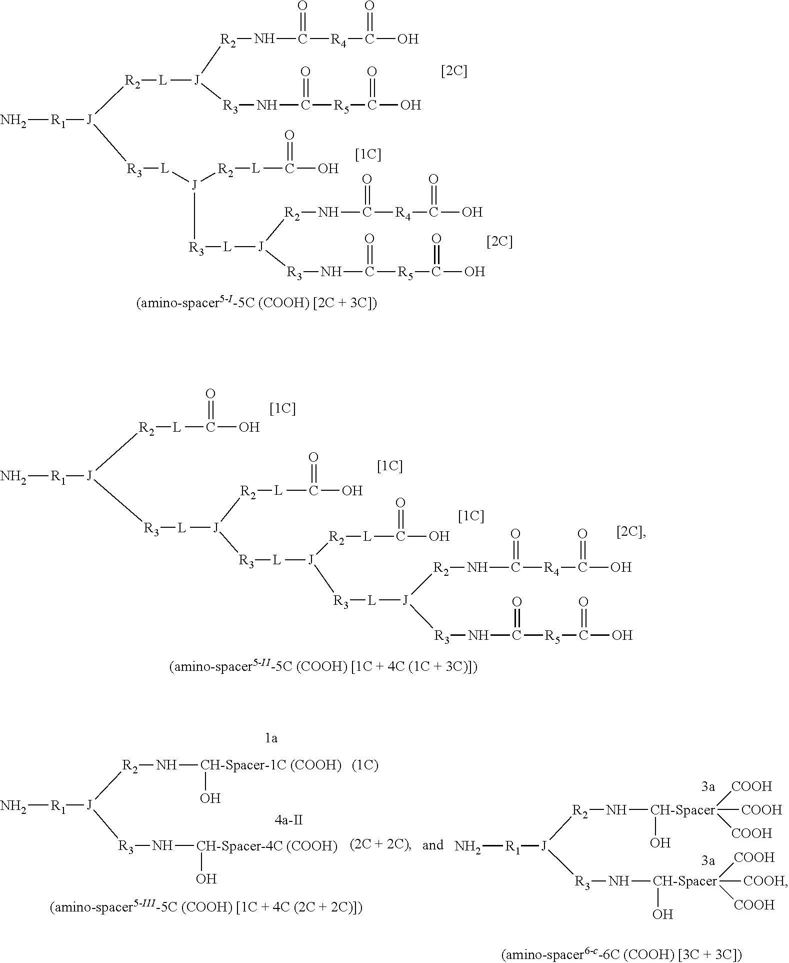

wherein
each R₁, R₂, and R₃ is independently C₁₋₂₀ alkylene, C₁₋₆ alkylene oxide, or polyalkylene oxide;
each R₄ and R₅ is independently C₁₋₂₀ alkylene;
each J is CH, N, or NH;
each J₁ is independently C₁₋₂₀ alkylene, C₁₋₆ alkylene oxide, or polyalkylene oxide;

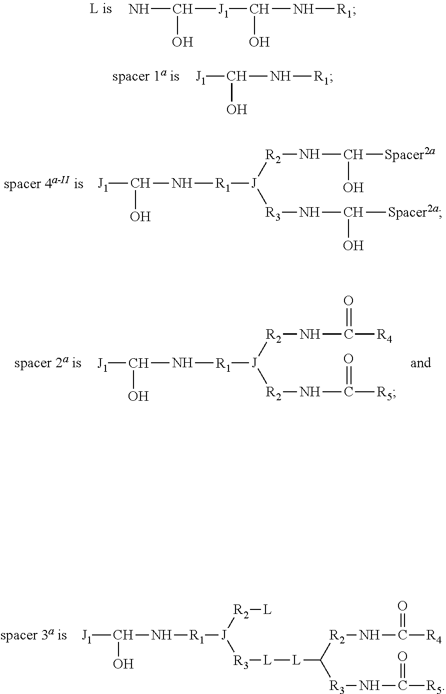

The term “alkylene” used herein refers to an alkyl group (i.e., straight chained and branched saturated hydrocarbon groups containing one to twenty carbon atoms) having a substituent. For example, an alkylene group can be —CH₂CH₂— or —CH₂—. The term Cn means the alkylene group has “n” carbon atoms. For example, C₁₋₂₀ alkylene refers to an alkylene group having a number of carbon atoms encompassing the entire range, as well as all subgroups (e.g., 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, 1-2, 1, 2, 3, 4, 5, 6, 7, 8, 9, 10, 11, 12, 13, 14, 15, 16, 17, 18, 19, and 20 carbon atoms). Unless otherwise indicated, an alkylene group can be an unsubstituted alkylene group or a substituted alkylene group.

The term “alkylene oxide” used herein refers to an —O-alkylene group. For example, an alkylene oxide group can be —OCH₂CH₂— or —CH₂O—. The term Cn means the alkylene oxide group has “n” carbon atoms. For example, C₁₋₆ alkylene oxide refers to an alkylene oxide group having a number of carbon atoms encompassing the entire range, as well as all subgroups, as previously described for “alkylene” groups.

The term “polyalkylene oxide” used herein refers to polymers or copolymers of alkylene oxides. Nonlimiting examples of polyalkylene oxides include polyethylene oxide (PEO), polypropylene oxide (PPO), and polybutylene oxide. Polyalkylene oxides can have a molecular weight of 200 g/mol or greater, e.g., 200 g/mol to 20,000 g/mol.

In some embodiments, at least one R₁ is C₁₋₂₀ alkylene. In some embodiments, each R₁ is C₁₋₂₀ alkylene. In some embodiments, at least one R₁ is C₁₋₆ alkylene oxide. In some embodiments, each R₁ is C₁₋₆ alkylene oxide. In some embodiments, at least one R₁ is polyalkylene oxide. In some embodiments, each R₁ is polyalkylene oxide.

In some embodiments, at least one R₂ is C₁₋₂₀ alkylene. In some embodiments, each R₂ is C₁₋₂₀ alkylene. In some embodiments, at least one R₂ is C₁₋₆ alkylene oxide. In some embodiments, each R₂ is C₁₋₆ alkylene oxide. In some embodiments, at least one R₂ is polyalkylene oxide. In some embodiments, each R₂ is polyalkylene oxide.

In some embodiments, at least one R₃ is C₁₋₂₀ alkylene. In some embodiments, each R₃ is C₁₋₂₀ alkylene. In some embodiments, at least one R₃ is C₁₋₆ alkylene oxide. In some embodiments, each R₃ is C₁₋₆ alkylene oxide. In some embodiments, at least one R₃ is polyalkylene oxide. In some embodiments, each R₃ is polyalkylene oxide.

In some embodiments, at least one J is CH. In some embodiments, each J is CH. In some embodiments, at least one J is N. In some embodiments, each J is N.

In some embodiments, at least one J₁ is C₁₋₂₀ alkylene. In some embodiments, each J₁ is C₁₋₂₀ alkylene. In some embodiments, at least one J₁ is C₁₋₆ alkylene oxide. In some embodiments, each J₁ is C₁₋₆ alkylene oxide. In some embodiments, at least one J₁ is polyalkylene oxide. In some embodiments, each J₁ is polyalkylene oxide.

In some embodiments, C₁₋₆ alkylene oxide is ethylene oxide or propylene oxide. In some embodiments, C₁₋₆ alkylene oxide is ethylene oxide. In some embodiments, C₁₋₆ alkylene oxide is propylene oxide.

In some embodiments, polyalkylene oxide is polyethylene oxide (PEO) or polypropylene oxide (PPO). In some embodiments, polyalkylene oxide is polyethylene oxide (PEO). In some embodiments, polyalkylene oxide is polypropylene oxide (PPO). In some embodiments, the polyethylene oxide or polypropylene oxide has an average molecular weight of 200 g/mol or greater. In some embodiments, the polyethylene oxide or polypropylene oxide has an average molecular weight of 200 g/mol to 20,000 g/mol.

### Amino-Spacerⁿ-n Carboxyl (COOH) Moieties

The biological assay devices disclosed herein comprise a reactive solid substrate linked to one or more amino-spacerⁿ-n Carboxyl (COOH) moieties. The amino-spacerⁿ-n Carboxyl (COOH) moieties can comprise two or more branches derived from combinations of initial amino-spacerⁱ-i C (COOH), epoxy-spacer^{m}-m C (COOH), bifunctional epoxy compounds, and multifunctional amines, wherein i and m are each independently integers from 1 to 20.

As used herein the term “multifunctional amine” refers to compounds of general structure:

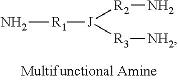

wherein R₁, R₂, R₃, and J are as described herein.

In some embodiments, the amino-spacerⁿ-n Carboxyl (COOH) moieties disclosed herein one or more nitrogen atoms comprise a multifunctional amine-derived moiety. Non-limiting examples of multifunctional amine-derived moieties include a moiety derived from amino dextran, polyethylenimine, poly (vinyl amine), poly (allyl amine), poly (ethylene glycol), or poly (propylene glycol) based trifunctional amines.

Nonlimiting examples of amino-spacerⁿ-n Carboxyl (COOH) moieties and the components from which they can be made are discussed herein below.

### Amino-Spacer¹-1 C (COOH)

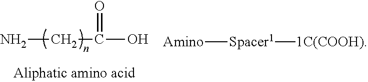

Spacer¹: (CH₂)ₙ, n=1-20. Amino-spacer¹-1 C (COOH) is used to generate amino-spacerⁿ-n C (COOH) moieties with n>1.

### Amino-Spacer²-2 C (COOH)

The structure of amino-spacer²-2 C (COOH) is shown below.

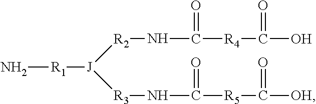

wherein R₁, R₂, R₃, R₄, R₅, and J are as described herein.

The structure of amino-spacer²-2 C (COOH) can also be simplified as shown below.

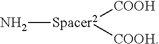

The structure of spacer² is shown below.

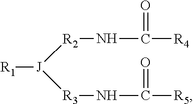

wherein R₁, R₂, R₃, R₄, R₅, and J are as described herein.

The structure of amino-spacer²-2 C (COOH) can be simplified as shown below.

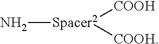

As used herein the term “bifunctional epoxy compound” refers to compounds of general structure:

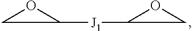

wherein J₁ is as described herein.

The general structure of a diglycidyl ether is shown below.

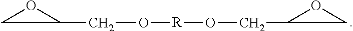

Particularly contemplated are polyethylene glycol diglycidyl ethers having the general structure:

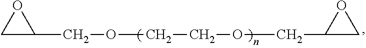

wherein n is an integer from 1 to 1,000. Nonlimiting examples of diglycidyl ethers include ethylene glycol diglycidyl ether, poly (ethylene glycol) diglycidyl ether, and poly (propylene glycol) diglycidyl ether.
Modification of Surfaces with Amino-Spacerⁿ-n Carboxyl (COOH) Moieties

The biological assay devices disclosed herein comprise a reactive solid substrate linked to one or more amino-spacerⁿ-n Carboxyl (COOH) moieties. In some embodiments, the solid substrates comprise an epoxy resin. In some embodiments, the epoxy resin is SU-8. SU-8 is an epoxy composed of bisphenol A novolac epoxy that is dissolved in an organic solvent (gamma-butyrolactone GBL or cyclopentanone, depending on the formulation) and up to 10 wt % of mixed triarylsulfonium/hexafluoroantimonate salt as the photoacid generator.

Suitable solid substrates include films, microporous membranes, beads, magnetic beads, barcoded magnetic beads, and nickel barcoded magnetic beads. In some embodiments, the barcoded magnetic beads comprise a series of alternating light transmissive and opaque sections, with relative positions, widths and spacing resembling a 1D or 2D bar code image. In some embodiments, the opaque sections comprise a paramagnetic material. In some embodiments, the paramagnetic material is Ni.

The solid substrates described herein can be reactive solid substrates, i.e., solid substrates comprising one or more reactive functionalities selected from epoxy, amino, carboxyl, and thiol groups. In some embodiments, the epoxy groups comprise ethylene glycol diglycidyl ether, poly (ethylene glycol) diglycidyl ether, or poly (propylene glycol) diglycidyl ether.

In some embodiments, the reactive solid substrate comprises an epoxy-containing SU-8 barcoded magnetic bead. A scheme showing the linking of an epoxy-containing SU-8 barcoded magnetic bead with one or more amino-spacerⁿ-n Carboxyl (COOH) moieties is presented below.

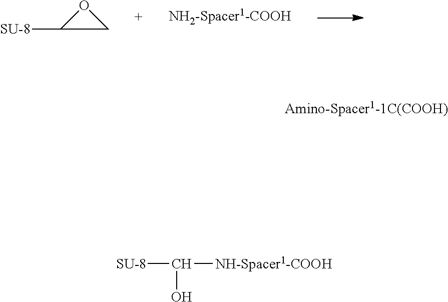

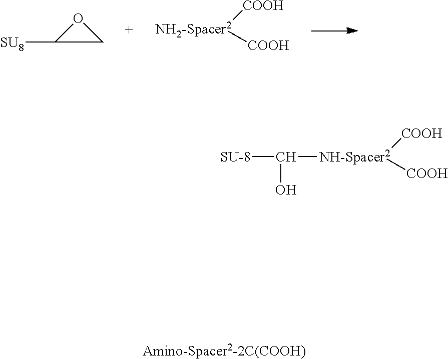

Each reactive epoxy group on bead surface reacting with amino group in Amino-Spacer¹-1 C (COOH) can produce one carboxyl group on SU-8 barcoded magnetic beads surface. Each reactive epoxy group on bead surface reacting with Amino-Spacer²-2 C (COOH) can produce two carboxyl functionalities on SU-8 barcoded magnetic beads surface.

The following new process was designed to produce more carboxyl functionalities on barcoded magnetic beads.

The preparation of new surface modifying reagents containing an amino group at one end and n C (carboxyl) at the other end required four components, such as amino-Spacerⁿ-n C (COOH) (n≥1), epoxy-spacer^{ma}-m C (COOH) (m≥1), a bifunctional epoxy compound, such as ethylene glycol diglycidyl ether, poly (ethylene glycol) diglycidyl ether, or poly (propylene glycol) diglycidyl ether, and a multifunctional amine, such as a tris(aminoalkyl) amine, poly(ethylene glycol) or poly(propylene glycol) based multifunctional amine. The resulting new modifying reagent has a designed branched spacer structure between the amino and n C (carboxyl) ends.

Four components are required to synthesize new reagents for surface modification of reactive substrates which can be used for bioassays of biological molecules. An overview of these components with examples of each is shown in Table 1.

TABLE 1

| Component (I) | Amino-Spacer ¹-1 C (COOH) |
|---|---|
| Amino-Spacer ⁿ-n C (COOH) | Amino-Spacer ²-2 C (COOH) |
| n ≥ 1 | Amino-Spacer ³-3 C (COOH) |
| | Amino-Spacer ⁴-4 C (COOH) |
| | Amino-Spacer ⁵-5 C (COOH) |
| | Amino-Spacer ⁶-6 C (COOH) |
| | etc. |
| Component (II) | Epoxy-Spacer ^{1a}-1 C (COOH) |
| Epoxy-Spacer ^{ma}-m C (COOH) | Epoxy-Spacer ^{2a}-2 C (COOH) |
| m ≥ 1 | Epoxy-Spacer ^{3a}-3 C (COOH) |
| | Epoxy-Spacer ^{4a}-4 C (COOH) |
| | Epoxy-Spacer ^{5a}-5 C (COOH) |
| | Epoxy-Spacer ^{6a}-6 C (COOH) |
| | etc. |
| Component (III) | Trifunctional amine |
| | Tris (amino alkyl) amine |
| Multifunctional amines | Poly (ethylene glycol) based triamine |
| | Poly (propylene glycol) based triamine |
| | Polyethylenimine |
| | Poly (vinyl amine) |
| | Poly (allyl amine) |
| | Amino dextran |
| | etc. |
| Component (IV) | Ethylene glycol diglycidyl ether |
| Bifunctional epoxy compounds | 1,4-Butanediol diglycidyl ether |
| | 1,6-Hexanediol diglycidyl ether |
| | Poly (ethylene glycol) diglycidyl ether |
| | Poly (propylene glycol) diglycidyl |
| | ether etc. |
| Note: | |

^{a}is used to distinguish between amino-spacer ⁿ-n C (COOH) and epoxy-
spacer ^{ma}-m C (COOH). The designation of any spacer related to epoxy
will contain a, and the designation of any spacer related to amino will
contain no ^{a}.

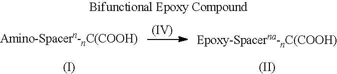

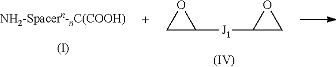

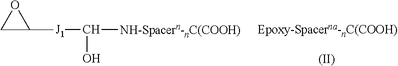

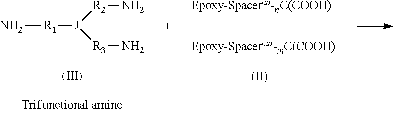

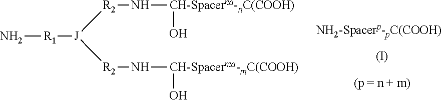

An amino-spacerⁿ-n C (COOH) (1) can be converted into an epoxy-spacer^{na}-n C (COOH) (II) by reacting with the same equivalent of bifunctional epoxy compound (IV).

Table 2 shows that amino-spacer^{p}-α C (COOH) (p≥2) can be prepared by reacting trifunctional amine (1 equiv.) with the mixture of epoxy-spacer^{na}-n C (COOH) (1 equiv.) and epoxy-spacer^{ma}-m C (COOH) (1 equiv.) in various combinations.

TABLE 2
p = n + m (n ≥ 1, m ≥ 1) p ≥ 2

| n | m | p |
|---|---|---|
| 1 | 1 | 2 |
| 1 | 2 | 3 |
| 1 | 3 | 4 |
| 2 | 2 | 4 |
| 1 | 4 [1 + 3] | 5 |
| 1 | 4 [2 + 2] | 5 |
| 2 | 3 [1 + 2] | 5 |
| 1 | 5 [1 + 4 (1 + 3)] | 6 |
| | 5 [1 + 4 (2 + 2)] | |
| | 5 [2 + 3] | |
| 2 | 4 [1 + 3] | 6 |
| | 4 [2 + 2] | |
| 3 | 3 | 6 |

Amino-spacer^{p}-p C (COOH) (I) can be converted into epoxy-spacer^{pa}-p C (COOH) (II) by reacting with same equivalent of bifunctional epoxy compound (IV).

Table 3 shows amino-spacer^{r}-r C (COOH) (r≥3) (1) can be prepared by reacting trifunctional amine (1 equiv.) (III) with the mixture of epoxy-spacer^{na}-n C (COOH) (1 equiv.) (II) and epoxy-spacer^{pa}-p C (COOH) (1 equiv.) (II) in various combinations.

TABLE 3
r = n + p (n ≥ 1, p ≥ 2); r ≥ 3

| n | p | r |
|---|---|---|
| 1 | 2 | 3 |
| 1 | 3 | 4 |
| 2 | 2 | 4 |
| 1 | 4 [1 + 3] | 5 |
| 1 | 4 [2 + 2] | 5 |
| 2 | 3 | 5 |
| 1 | 5 [1 + 4 (1 + 3)] | 6 |
| | 5 [1 + 4 (2 + 2)] | |
| | 5 [2 + 3] | |
| 2 | 4 [1 + 3] | 6 |
| | 4 [2 + 2] | |
| 3 | 3 | 6 |

Amino-spacer^{s}-s C (COOH) (s≥4) can be prepared by the same process. Amino-spacer^{t}-t C (COOH) (t≥5) can be prepared by the same process. Amino-spacer^{u}-u C (COOH) (u≥6) can be prepared by the same process.

Amino-spacer¹-1 C (COOH) can be converted into epoxy-spacer^{1a}-1 C (COOH) by reacting with same equivalent of bifunctional epoxy compound, as shown in the scheme below.

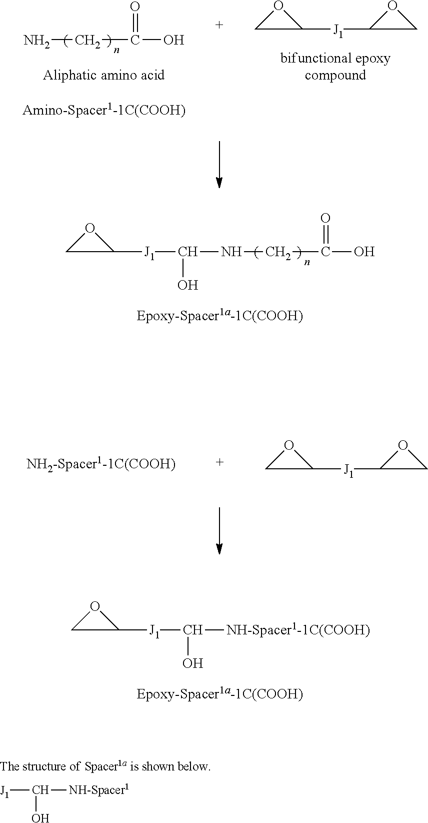

The synthesis of Epoxy-Spacer^{2a}-2 C (COOH) is shown in the scheme below.

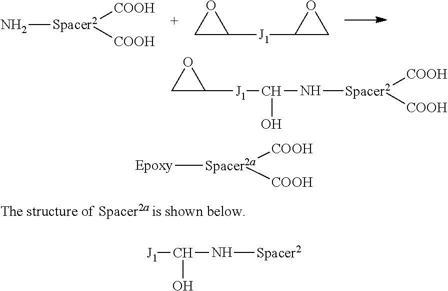

The synthesis of Amino-Spacer³-3 C (COOH) is shown in the scheme below.

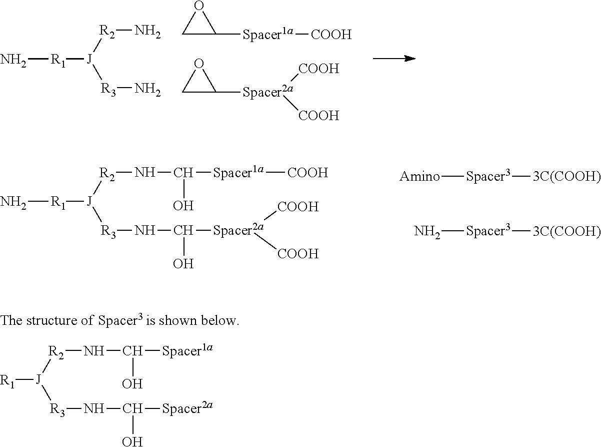

Without wishing to be bound by any particular theory, each epoxy group on SU-8 bead surface reacts with amino group in Amino-Spacer³-3 C (COOH) and generates three carboxyl (COOH) functionalities on the SU-8 bead surface.

The synthesis of Epoxy-Spacer^{3a}-3 C (COOH) is shown in the scheme below.

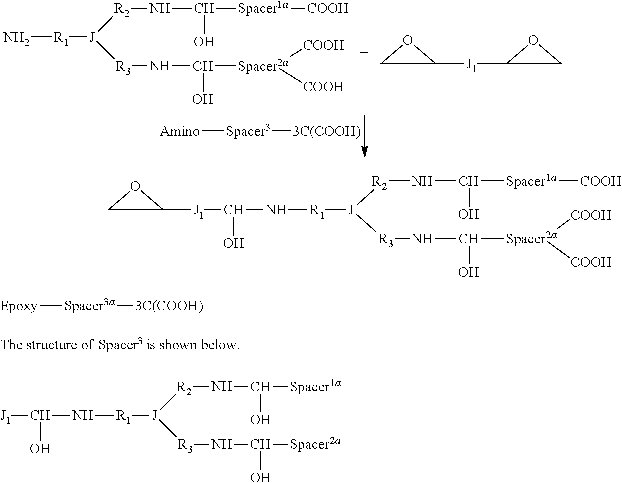

It can be simplified as shown in the below scheme.

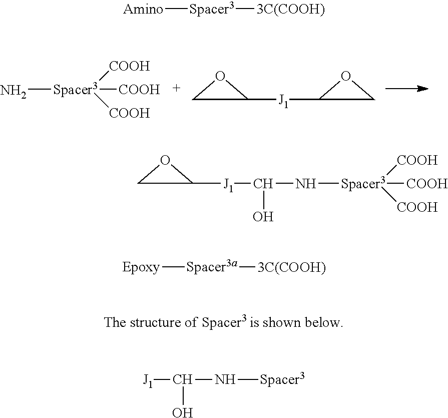

Amino-Spacer⁴-4 C (COOH) can be formed either by the reaction between 1 equiv. of trifunctional amine and (a) the mixture of 1 equiv. of epoxy-spacer^{1a}-1 C (COOH) and 1 equiv. of epoxy-spacer^{3a}-3 C (COOH) or (b) 2 equiv. of Epoxy-Spacer^{2a}-2 C (COOH).

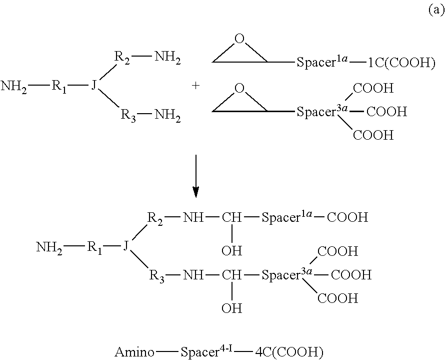

The structure of Spacer⁴-1 is shown below.

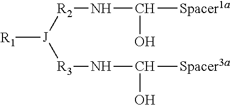

Without wishing to be bound by any particular theory, each epoxy group on SU-8 bead surface reacts with an amino group in amino-spacer^{4-I}-4 C (COOH) to generate four carboxyl (COOH) functionalities on the SU-8 bead surface.

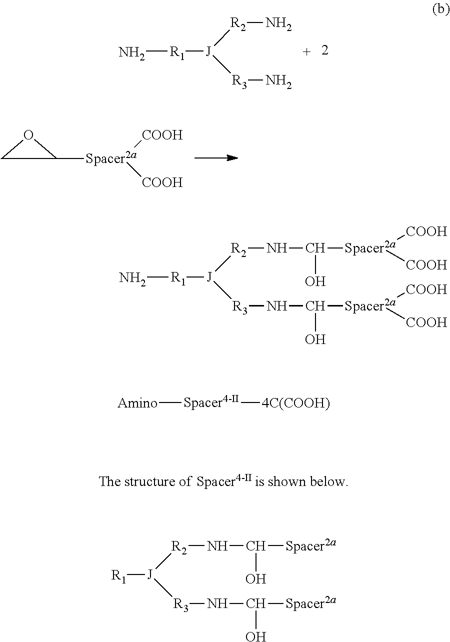

Without wishing to be bound by any particular theory, each epoxy group on SU-8 bead surface reacts with an amino group in amino-spacer^{4-II}-4 C (COOH) to generate four carboxyl (COOH) functionalities on SU-8 bead surface.

The epoxy-spacer^{4a-I}-4 C (COOH) can be prepared by reacting amino-spacer^{4-I}-4 C (COOH) with same equivalent of bifunctional epoxy compound.

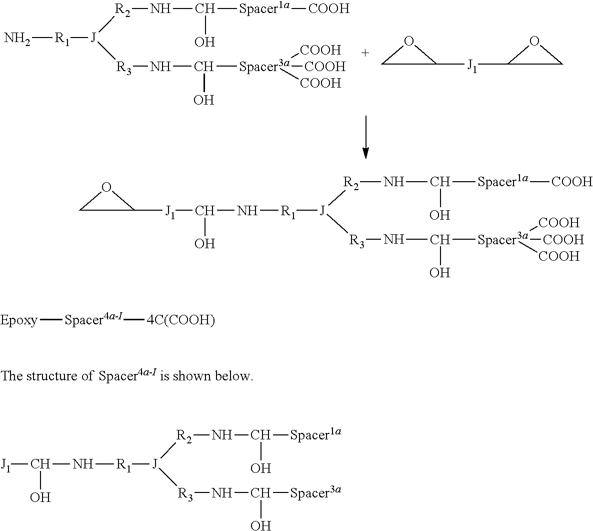

Epoxy-spacer^{4a-II}-4 C (COOH) can be prepared by reacting amino-spacer^{4-II}-4 C (COOH) with the same equivalent of bifunctional epoxy compound, as shown in the scheme below.

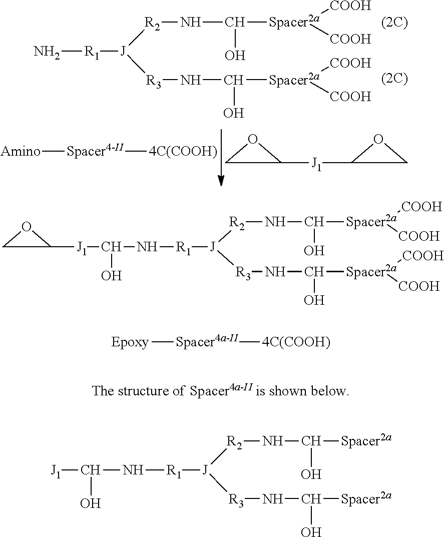

Amino-spacer⁵-5 C (COOH) can be prepared by reacting 1 equiv. of trifunctional amine with (a) a mixture of 1 equiv. of epoxy-spacer²-2 C (COOH) and 1 equiv. of epoxy-spacer^{3a}-3 C (COOH) [1 C+2 C], or (b) a mixture of 1 equiv. of epoxy-spacer^{1a}-1 C (COOH) and 1 equiv. of epoxy-spacer^{4a-I}-4 C (COOH) [1 C+3 C], or (c) a mixture of 1 equiv. of epoxy-spacer^{1a}-1 C (COOH) and 1 equiv. of epoxy-spacer^{4a-II}-4 C (COOH) [2 C+2 C].

Reaction (a) is shown in the scheme below.

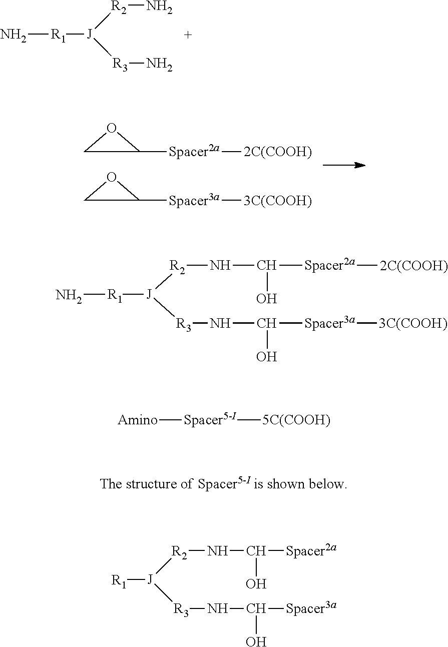

Without wishing to be bound by any particular theory, each epoxy group on the SU-8 bead surface reacts with an amino group in amino-spacer^{5-I}-5 C (COOH) to generate five carboxyl (COOH) functionalities on SU-8 bead surface.

Reaction (b) is shown in the scheme below.

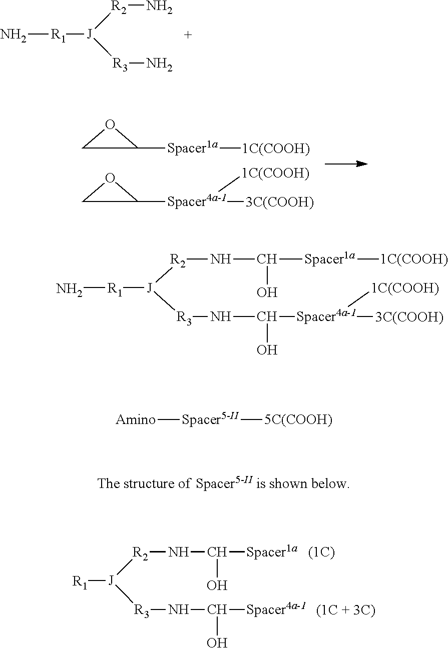

Without wishing to be bound by any particular theory, an epoxy group on the SU-8 bead surface reacts with an amino group in amino-spacer^{5-II}-5 C (COOH) to generate five carboxyl (COOH) functionalities on SU-8 bead surface.

Reaction (c) is shown in the scheme below.

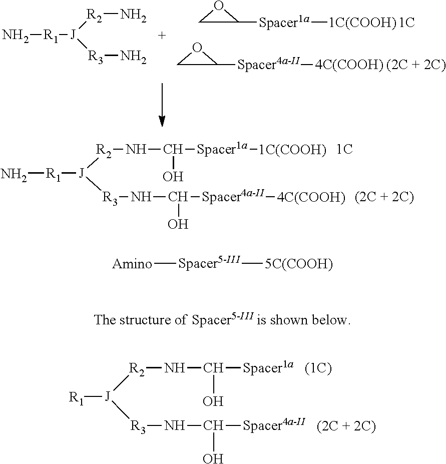

Without wishing to be bound by any particular theory, an epoxy group on the SU-8 bead surface reacts with an amino group in amino-spacer^{5-III}-5 C (COOH) to generate five carboxyl (COOH) functionalities on SU-8 bead surface.

Epoxy-Spacer^{5a-I}-5 C (COOH) (2 C+3 C) can be prepared as shown in the scheme below.

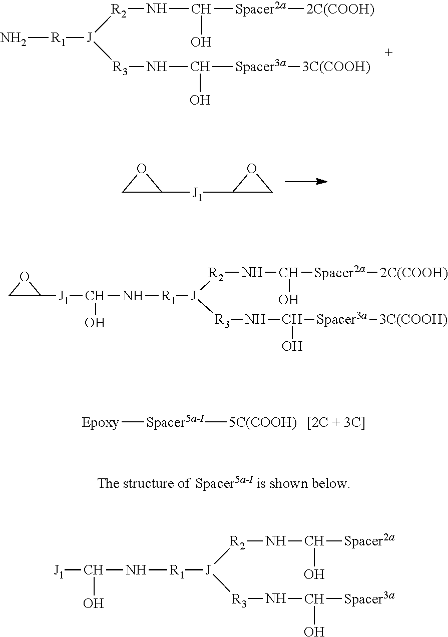

Epoxy-Spacer^{5a-II}-5 C (COOH) [1 C+4 C (1 C+3 C)] can be prepared as shown in the scheme below.

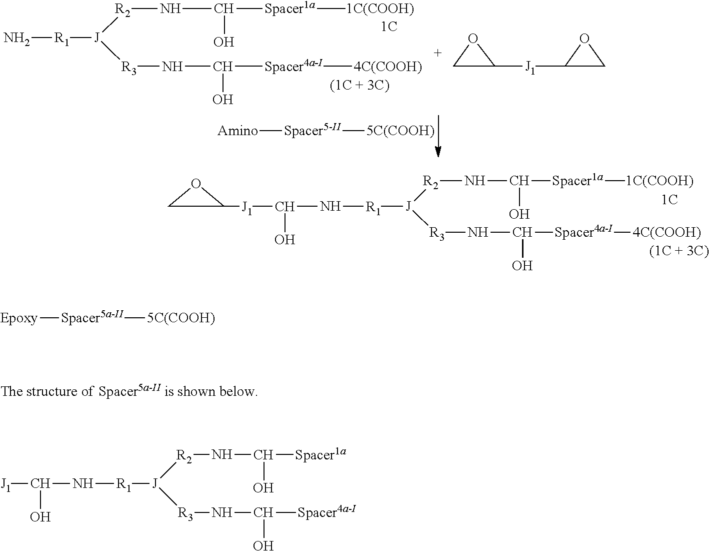

Epoxy-Spacer^{5a-III}-5 C (COOH) [1 C+4 C (2 C+2 C)] can be prepared as shown in the scheme below.

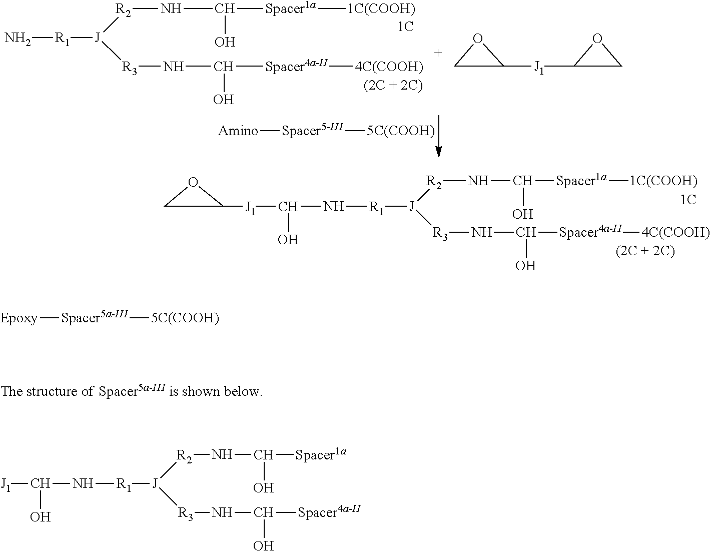

Amino-Spacer⁶-6 C (COOH) can be prepared by reacting 1 equiv. of trifunctional amine with various epoxy-spacers, including (a) 1 equiv. of epoxy-spacer^{1a}-1 C (COOH) and 1 equiv. of epoxy-spacer^{5a-I}-5 C (COOH) (2 C+3 C) [yielding Amino-Spacer^{6-(a)-I}-6 C (COOH)], or epoxy-spacer^{5a-I}-5 C (COOH) [1 C+4 C (1 C+3 C)] [yielding Amino-Spacer^{6-(a)-II}-6 C (COOH)], or epoxy-spacer^{5a-III}-5 C (COOH) [1 C+4 C (2 C+2 C)] [yielding Amino-Spacer^{6-(a)-III}-6 C (COOH)]; (b) 1 equiv. of epoxy-spacer^{2a}-2 C (COOH) and 1 equiv. of epoxy-spacer^{4a-I}-4 C (COOH) (1 C+3 C) [yielding Amino-Spacer^{6-(b)-I}-6 C (COOH)], or epoxy-spacer^{4a-II}-4 C (COOH) (2 C+2 C) [yielding Amino-Spacer^{6-(b)-II}-6 C (COOH)]; or (c) 2 equiv. of epoxy-spacer^{3a}-3 C (COOH) (1 C+2 C) [yielding Amino-Spacer^{6-(c)}-6 C (COOH)].

The reaction for case (c) is shown in the scheme below.

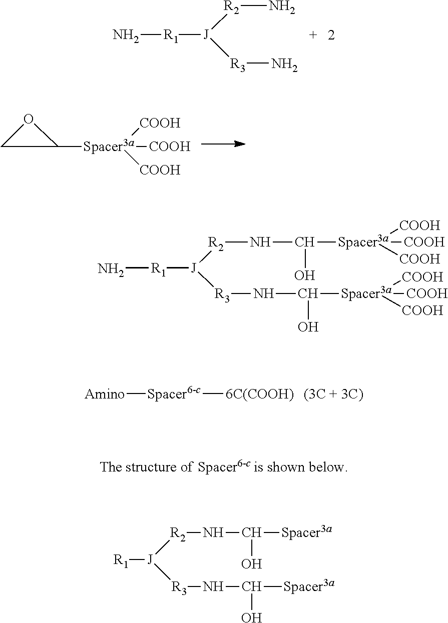

Without wishing to be bound by any particular theory, an epoxy group on the SU-8 bead surface reacts with an amino group in amino-spacer^{6-c}-6 C (COOH) to generate six carboxyl (COOH) functionalities on SU-8 bead surface.

The various amino-spacer⁶-6 C (COOH) combinations described above can be converted into epoxy-spacer^{6a}-6 C (COOH) by reacting with the same equiv. of bifunctional epoxy compounds as shown in the scheme below.

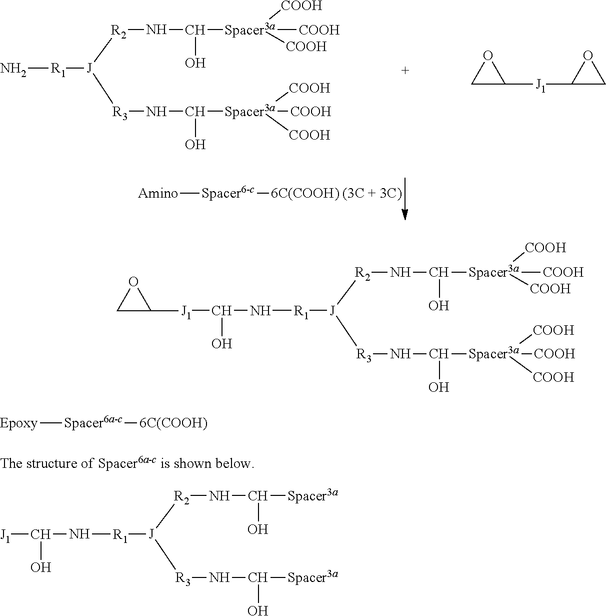

Other amino-spacerⁿ-n C (COOH) (n>1) or epoxy-spacer^{ma}-m C (COOH) (m>1) moieties can be prepared by any appropriate combination of amino-spacer^{p}-p C (COOH) (p≥1), epoxy-spacer^{qa}-q C (COOH) (q≥1), multifunctional amine (trifunctional amine), and diglycidyl ether (e.g., ethylene glycol diglycidyl ether, poly(ethylene glycol) diglycidyl ether, or poly(propylene glycol) diglycidyl ether).

The configuration of the amino-spacerⁿ-n C (COOH) (n>1) or epoxy-spacer^{ma}-m C (COO H) (m>1) moieties disclosed herein may resemble fish bones or tree branches. Each branch in the amino-spacerⁿ-n C (COOH) (n>1) or epoxy-spacer^{ma}-m C (COO H) (m>1) moieties disclosed herein can undergo a specific bio-interactions.

In general, the amino-spacerⁿ-n Carboxyl (COOH) moieties described herein may be prepared by admixing one or more initial amino-spacerⁱ-i C (COOH) moieties, one or more epoxy-spacer^{m}-m C (COOH) moieties, one or more multifunctional amines, and/or one or more diglycidyl ethers of C₁₋₆ alkylene oxides or polyalkylene oxides, wherein n is an integer from 2 to 6, and i and m are each independently integers from 1 to 20. In some embodiments, the one or more multifunctional amines comprise one or more trifunctional amines. Persons skilled in the art will be able to select appropriate starting materials and conditions to synthesize any of the amino-spacerⁿ-n Carboxyl (COOH) moieties described herein in view of the preceding discussion and the Examples set forth below.

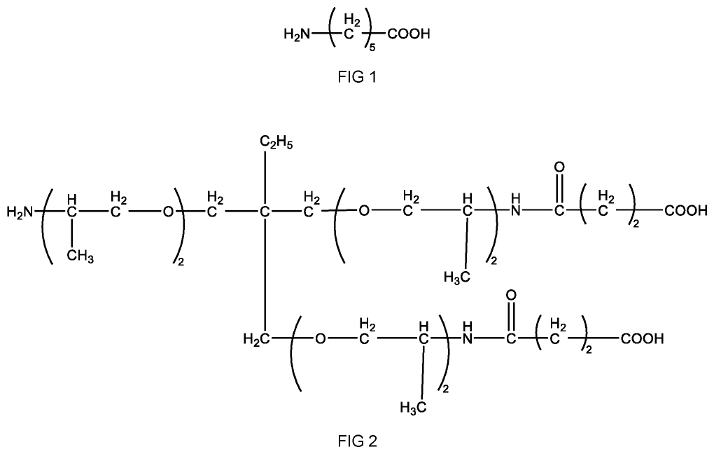

Specific, non-limiting examples of amino-spacerⁿ-n Carboxyl (COOH) moieties disclosed herein are shown in the attached FIGS. 1-6, as described in Table 4.

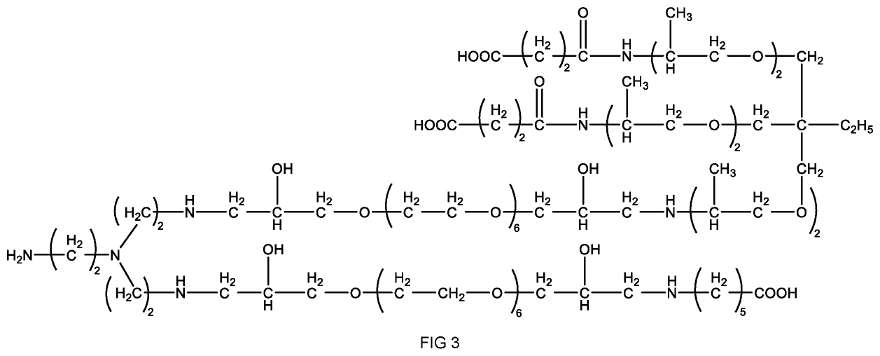

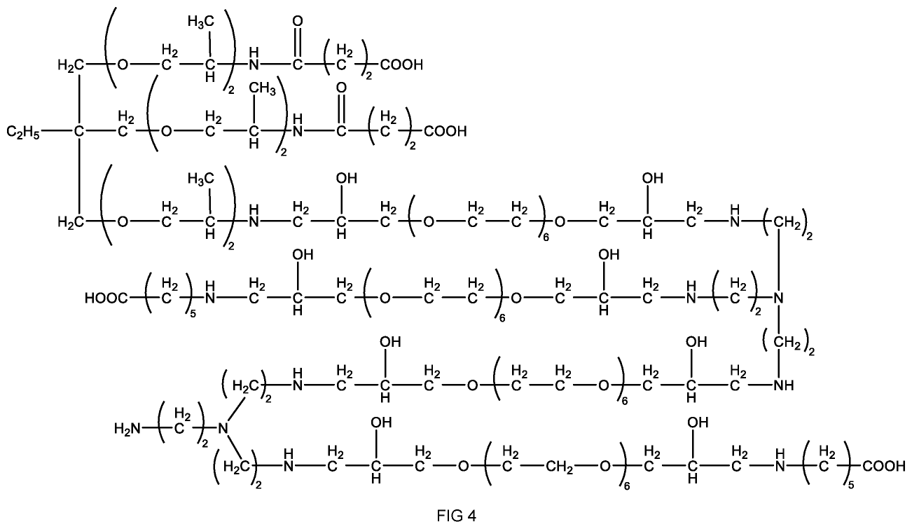

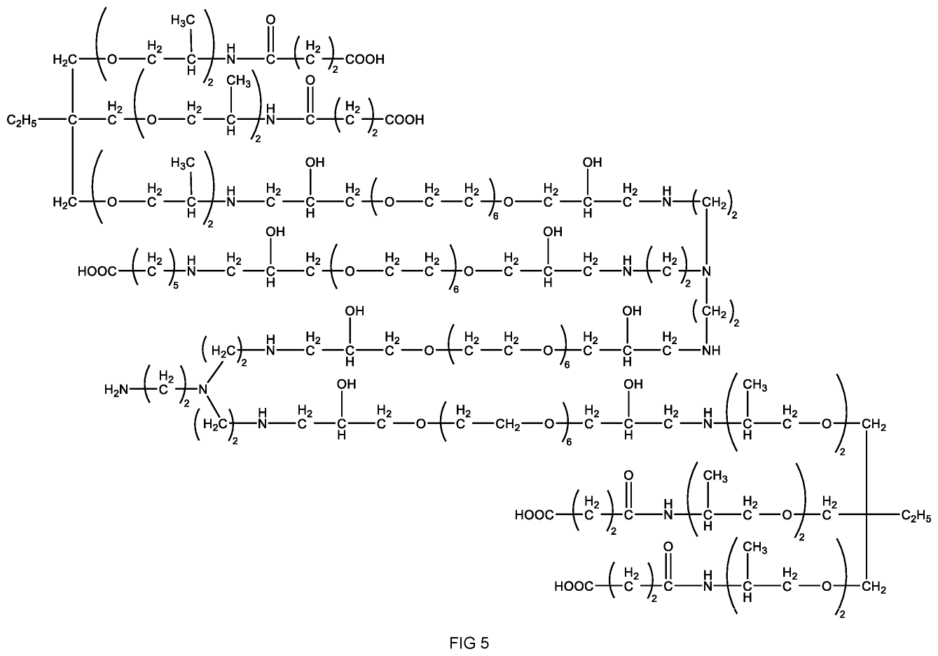

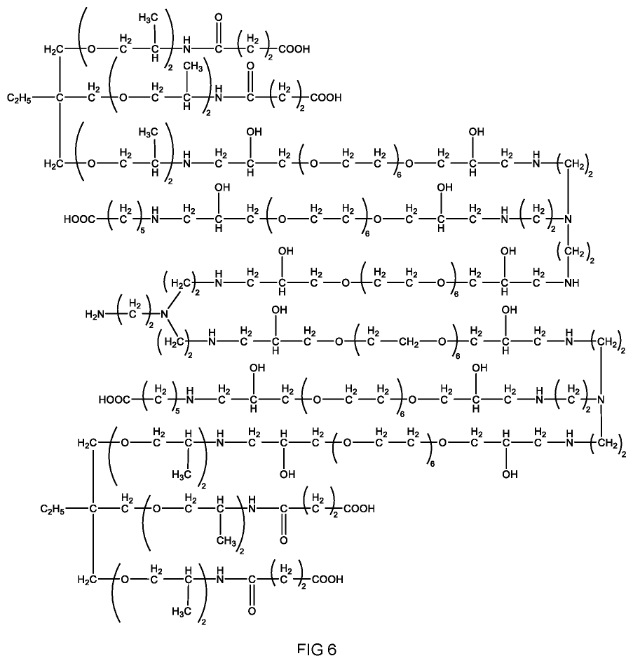

TABLE 4

| Compound | FIG |
|---|---|
| Compound 1 (Amino-Spacer¹ -1 C) | FIG. 1 |
| Compound 2 (Amino-Spacer² -2 C) | FIG. 2 |
| Compound 3 (Amino-Spacer³ -3 C) | FIG. 3 |
| Compound 4 (Amino-Spacer⁴ -4 C) | FIG. 4 |
| Compound 5 (Amino-Spacer⁵ -5 C) | FIG. 5 |
| Compound 6 (Amino-Spacer⁶ -6 C) | FIG. 6 |

### Biological Assays

The biological assay devices described herein are useful for the detection of various biomolecules of interest. The structures of the amino-spacerⁿ-n C (COOH) moieties incorporated into the biological assay devices described herein are carefully designed to enhance the efficiency to capture very low concentration of biomolecules of interest. Without wishing to be bound by any particular theory, increased surface functionalities on the bead surface can increase the sensitivity of tests using the biological assay devices disclosed herein using relatively small quantities of biological probe molecules.

Non-limiting examples of biological probe molecules that can be used with the biological assay devices described herein include antibodies, proteins, oligopeptides, peptide sequences, and oligonucleosides, DNA, and RNA.

Without wishing to be bound by any particular theory, increased surface functionalities on the bead surface also can increase the binding capacity of biomolecules (e.g., biological probe molecules) using a relatively large quantity of probes due to the controlled structure of the amino-spacerⁿ-n C (COOH) moieties incorporated into the biological assay devices described herein.

In some embodiments, and as illustrated in the Examples set forth below, the biological assay devices disclosed herein exhibit a higher fluorescence intensity (MFI) after coupling with fluorescent biological probes than biological assay devices that do not comprise one or more amino-spacerⁿ-n Carboxyl (COOH) moieties. In some embodiments, the biological assay devices show higher fluorescence intensity (MFI) and lower background fluorescence after coupling with fluorescent biological probes than biological assay devices that do not comprise one or more amino-spacerⁿ-n Carboxyl (COOH) moieties. In some embodiments, the biological assay devices show a higher dynamic range after coupling with biological probes than biological assay devices that do not comprise one or more amino-spacerⁿ-n Carboxyl (COOH) moieties.

The biological assay devices disclosed herein are useful for methods of detecting a substance in a biological sample. In some embodiments, the methods comprise contacting the biological sample with a biological assay device disclosed herein. In some embodiments, the substance is an antigen, antibody, virus, pathogen, DNA, or RNA.

### EXAMPLES

### General Synthetic Procedures

1. Epoxy-spacer^{1a}-1 C (COOH) was prepared by mixing 1 equiv. of aliphatic amino acid [amino-spacer¹-1 C (COOH)] and 1 equiv. of a bifunctional epoxy compound, such as polyethylene glycol) diglycidyl ether or poly(propylene glycol) diglycidyl ether and heating at 45-60° C. for 1-3 hours to form 1 equiv. of epoxy-spacer^{1a}-1 C (COOH).

2. Epoxy-spacer^{2a}-2 C (COOH) was prepared by mixing 1 equiv. of reaction product between trifunctional amine and succinic anhydride or maleic anhydride or glutaric anhydride [amino-spacer²-2 C (COOH)] and 1 equiv. of a bifunctional epoxy compound, such as polyethylene glycol) diglycidyl ether or poly(propylene glycol) diglycidyl ether and heating at 45-60° C. for 1-3 hours to form 1 equiv. of epoxy-spacer^{2a}-2 C (COOH).

3. SU-8 standard BMB (50 K-1,000 K) beads were added to a 15 ml tube containing 12 mL of amino-spacer¹-1 C (COOH) solution or amino-spacer²-2 C (COOH) solution, then inserted into holder of rotator, and kept in an oven at 45-60° C. for 1-5 days. The modified SU-8 BMB beads were washed with diluted Tween-20 solution for several times, then stored in 1×PBS-T storage buffer.

4. A mixture of 1 equiv. of trifunctional amine and 1 equiv. of epoxy-spacer^{1a}-1 C (COOH) and 1 equiv. of epoxy-spacer^{2a}-2 C (COOH) was heated at 45-60° C. for 1-3 hours to form 1 equiv. of Amino-Spacer³-3 C (COOH). SU-8 standard BMB (50 K-1,000 K) beads were added to a 15 ml tube containing 12 mL of amino-pacer 3-3 C (COOH) solution, then inserted into holder of rotator, kept in an oven at 45-60° C. for 1-5 days. The modified SU-8 BMB beads were washed with diluted Tween-20 solution for several times, then stored in 1×PBS-T storage buffer.

5. A mixture of 1 equiv. of trifunctional amine and the mixture of any combination between 1 equiv. of epoxy-spacer^{na}-n C (COOH) (n≥1) and 1 equiv. of epoxy-spacer^{ma}-m C (COOH) (m≥1) was heated at 45-60° C. for 1-3 hours to form 1 equiv. of amino-spacer^{p}-p C (COOH) (p≥4). (Notes: n=1 & m≥3, or n≥3 & m=1, or n≥2 & m≥2; p=n+m)

SU-8 standard BMB (50 K-1,000 K) beads were added to a 15 ml tube containing 12 mL of amino-spacer^{p}-p C (COOH) (p≥4) solution, then inserted into holder of rotator, kept in an oven at 45-60° C. for 1-5 days. The modified SU-8 BMB beads were washed with diluted Tween-20 solution for several times, then stored in 1×PBS-T storage buffer.

### Oligo Probe Coupling Procedure

C-BMB tubes were quick spun and placed into a magnetic stand for 1 to 2 minutes. The supernatant was removed, and the C-BMB tubes were washed twice—with 200 μL of coupling buffer made from MES monohydrate (1-5%) in nuclease free water. To this was added 79 μL of coupling buffer, 1.0 μL of 100 μM amino-modified oligo probe (100 μmol), and 20 μL of 10 mg/mL EDC into the C-BMB/Probe mix tube. The tube was placed in a shaker for 2 hours and mixed at 1400 to 1600 rpm at room temperature.

The supernatant was removed, and 200 μL of TRIS Buffer was added. The tube was placed in a shaker for 15 to 20 minutes and mixed at 1400 to 1600 rpm at room temperature.

The BMB-probe was washed once with 200 UL of blocking buffer (Thermo Scientific-SuperBlock™ Blocking Buffer in PBS), then the BMB-probe was blocked with 200 μL of blocking buffer and shaken for 1 hour, mixed at 1400 to 1600 rpm, at room temperature.

Next, the blocking buffer was removed, and the BMB-probe was washed twice with 200 μL of PBST Buffer. The BMB-probe was stored in 200 μL of PBS-T buffer at 2 to 8° C. or used for the hybridization procedure.

### Hybridization Procedure

Approximately 50 beads/plex were used per well. The BMB-probe volume of each plex (analyte) was introduced into a master-mix tube. The master-mix tube was placed on the magnetic stand for 1 to 2 minutes, then the supernatant was removed using a pipette. A hybridization buffer made from TMAC (Tetramethylammonium chloride) (40-80% in nuclease-free water) was added to the master-mix tube, which was continuously mixed on a vortex until the beads were suspended. 48 UL BMB-probe suspension were then transferred into each well.

To this was added 2 μL of biotinylated target oligo probe into the corresponding wells. The covered BMB plate was placed in a shaker for 15 to 30 minutes, and mixed at 700 to 800 rpm at 52° C. Next, 5 g/ml SA-PE (a biotin-binding protein (streptavidin) covalently attached to a fluorescent label (R-Phycoerythrin)) in hybridization buffer was added. The BMB was washed with PBST, and then 50 μL of 5 μg/mL SA-PE were added per well. The covered BMB plate was placed in a shaker for 10 to 15 minutes, and mixed at 700 to 800 rpm at 52° C.

The BMB was washed 2 times with PBST, and the supernatant was removed. 250 μL of detection buffer comprising 20×SSC buffer, N-Lauroylsarcosine sodium and ACS reagent grade water was added to each well. The fluorescence intensity was then measured

### Example 1

GAPDH Coupling Amino-Spacer²-2 C (COOH) and Amino-Spacer³-3 C (COOH) modified SU-8 BMB and Hybridization with 3-fold serial diluted GAPDH Target, 25 min at 52° C., 820 rpm, SAPE 5 μg/mL, 12.5 min. A summary of the MFI observed for Compound 2 and Compound 3 with varying concentrations of GAPDH Target is presented in Table 5.

TABLE 5

| | Compound 2 | | | | | |
|---|---|---|---|---|---|---|
| GAPDH | (Amino- | | Compound 3 | | | |
| Target | Spacer² -2 C) | | (Amino-Spacer³ -3 C) | | | Control |
| fmol/μL | MFI | | MFI | | | MFI |
| 0.00 | 84 | 5 | 5 | 5 | 5 | 5 |
| 0.00026 | 14 | 2 | 2 | 22 | 2 | 2 |
| 0.00077 | 1 | 96 | 1 | 1 | 1 | 1 |
| 0.0023 | 73 | 69 | 37 | 69 | 41 | 5 |
| 0.0070 | 161 | 110 | 51 | 145 | 71 | 6 |
| 0.0021 | 627 | 591 | 603 | 525 | 538 | 3 |
| 0.063 | 1505 | 1465 | 1343 | 1354 | 1392 | 1 |
| 0.19 | 3934 | 3816 | 3927 | 3718 | 3706 | 3 |
| 0.56 | 10406 | 10520 | 11595 | 11015 | 10589 | 2 |
| 1.69 | 21671 | 22018 | 22895 | 20894 | 20768 | 7 |
| 5.08 | 35784 | 33916 | 35006 | 35641 | 33615 | 3 |
| 15.24 | 52235 | 50788 | 59195 | 56951 | 55115 | 4 |
| 45.72 | 81400 | 79795 | 136125 | 102825 | 86836 | 1 |
| 137.17 | 88253 | 91436 | 193722 | 157652 | 151894 | 3 |
| 411.52 | 96336 | 99749 | 193582 | 162167 | 163674 | 5 |
| 1234.57 | 105111 | 107989 | 193022 | 192902 | 191810 | 2 |

MFI: Fluorescence Intensity

Comparison between Amino-Spacer²-2 C (COOH) and Amino-Spacer³-3 C (COOH) modified SU-8 BMB, at low concentrations of GAPDH, Amino-Spacer²-2 C (COOH) modified BMB showed higher background (less sensitivity). At high concentrations of GAPDH, less surface functionalities on Amino-Spacer²-2 C (COOH) modified BMB surface resulted in lower MFI (fluorescence intensity). The Amino-Spacer²-2 C (COOH) modified SU-8 BMB already reached saturation capacity much lower than the instrument limit. Due to high efficiency to capture probes for Amino-Spacer³-3 C (COOH) modified SU-8 BMB with more carboxyl groups on surface, the modified BMB showed higher saturation capacity at higher concentration of GAPDH.

### Example 2

Various Amino-Spacerⁿ-n C (COOH) modified SU-8 BMB and Hybridization with 3-fold serial diluted GAPDH Target, 25 min at 52° C., 820 rpm, SAPE 5 μg/mL, 12.5 min. A summary of the MFI observed for Compound 2 and Compound 6 with varying concentrations of GAPDH Target is presented in Table 6.

TABLE 6

| | Compound 2 | Compound 6 | |
|---|---|---|---|
| GAPDH | (Amino- | (Amino- | Control |
| Target | Spacer² -2 C) | Spacer⁶ -6 C) | No treatment |
| fmol/μL | MFI | MFI | MFI |
| 0.00 | 1 | 65 | 1 |
| 0.00026 | 5 | 5 | 5 |
| 0.00077 | 198 | 159 | 7 |
| 0.0023 | 67 | 8 | 8 |
| 0.0070 | 115 | — | 6 |
| 0.0021 | 252 | 173 | 7 |
| 0.063 | 865 | 1045 | 4 |
| 0.19 | 2154 | 2986 | 5 |
| 0.56 | 6065 | — | 5 |
| 1.69 | 14545 | 16071 | 8 |
| 5.08 | 25154 | 28608 | 6 |
| 15.24 | 42901 | 49820 | 4 |
| 45.72 | 61293 | 120034 | 1 |
| 137.17 | 77215 | 194296 | 5 |
| 411.52 | 72943 | 193835 | 5 |
| 1234.57 | 83879 | 194135 | 3 |

Amino- Spacer⁶ - 6 C (COOH) modified SU-8 BMB show higher MFI (higher saturation capacity) than Amino- Spacer² - 2 C (COOH) modified SU-8 BMB.

### Example 3

Various Amino-Spacerⁿ-n C (COOH) modified SU-8 BMB and Hybridization with 3-fold serial diluted GAPDH Target, 25 min at 52° C., 820 rpm, SAPE 5 μg/mL, 12.5 min. A summary of the MFI observed for Compound 2, Compound 4, and Compound 6 with varying concentrations of GAPDH Target is presented in Table 7.

TABLE 7

| | Compound | Compound 4 | | | |
|---|---|---|---|---|---|
| GAPDH | 2(Amino- | (Amino- | Compound 6 (Amino- | | Control No |
| Target | Spacer² - | Spacer⁴ - | Spacer⁶ -6 C) | | treatment |
| fmol/μL | 2 C) MFI | 4 C) MFI | MFI | | MFI |
| 0.00 | 8 | 48 | 8 | 27 | 8 |
| 0.00026 | 186 | 117 | 2 | — | 7 |
| 0.00077 | 219 | 150 | 123 | 17 | 1 |
| 0.0023 | 372 | 190 | 257 | 90 | 7 |
| 0.0070 | 261 | 144 | — | 77 | 4 |
| 0.0021 | 457 | 457 | 323 | 303 | 2 |
| 0.063 | 1013 | 1144 | 1095 | 1294 | 8 |
| 0.19 | 2616 | 3256 | 2908 | 3594 | 4 |
| 0.56 | 8571 | 8853 | 8532 | 8395 | 8 |
| 1.69 | 17136 | 19589 | 18673 | 20923 | 1 |
| 5.08 | 31805 | 31383 | 34177 | 35160 | 1 |
| 15.24 | 44007 | 47684 | 54255 | 53988 | 7 |
| 45.72 | 68371 | 62521 | 179561 | 172819 | 3 |
| 137.17 | 64349 | 68250 | 194416 | 188093 | 7 |
| 411.52 | 60006 | 68758 | 194597 | — | 5 |
| 1234.57 | 70036 | 64599 | 194476 | 194476 | 6 |

Amino- Spacer⁶ - 6 C (COOH) modified SU-8 BMB showed higher MFI than amino- Spacer² - 2 C (COOH) or Amino- Spacer⁴ - 4 C (COOH) modified SU-8 BMB.

### Example 4

Various Amino-Spacer™-n C (COOH) modified SU-8 BMB and Hybridization with 3-fold serial diluted GAPDH Target, 25 min at 52° C., 820 rpm, SAPE 5 μg/mL, 12.5 min. A summary of the MFI observed for Compound 1, Compound 2, and Compound 3 with varying concentrations of GAPDH Target is presented in Table 8.

TABLE 8

| | Compound | Compound | | | | |
|---|---|---|---|---|---|---|
| GAPDH | 1 (Amino- | 2 (Amino- | | | | Control No |
| Target | Spacer¹ -1 | Spacer² -2 | Compound 3 (Amino- | | | treatment |
| fmol/μL | C) MFI | C) MFI | Spacer³ -3 C) MFI | | | MFI |
| 0.00 | 3 | 20 | 14 | 3 | 23 | 3 |
| 0.00026 | 14 | 215 | 57 | 72 | 51 | 4 |
| 0.00077 | 5 | 5 | 45 | 5 | 8 | 5 |
| 0.0023 | 35 | 116 | 133 | 108 | 90 | 6 |
| 0.0070 | 2 | 96 | 225 | 206 | 237 | 2 |
| 0.0021 | 6 | 116 | 485 | 335 | 309 | 6 |
| 0.063 | 23 | 625 | 1044 | 1085 | 958 | 5 |
| 0.19 | 166 | 1368 | 3020 | 2910 | 3013 | 8 |
| 0.56 | 565 | 3676 | 8761 | 8681 | 8301 | 3 |
| 1.69 | 1515 | 10829 | 18597 | 17925 | 17606 | 5 |
| 5.08 | 4305 | 20380 | 33460 | 32214 | 29811 | 5 |
| 15.24 | 9571 | 34062 | 48810 | 51909 | 45771 | 6 |
| 45.72 | 16615 | 50095 | 97401 | 85388 | 76091 | 3 |
| 137.17 | 22998 | 57244 | 191082 | 191082 | 191082 | 6 |
| 411.52 | 22268 | 73886 | 189926 | 189926 | 189926 | 5 |
| 1235 | 22531 | 78657 | 189533 | 189533 | 189533 | 1 |
| 3704 | 24830 | 78266 | 189036 | 189036 | 189036 | 7 |
| 11111 | 24983 | 70031 | 189705 | 189705 | 189705 | 2 |

For Amino-Spacer³ - 3 C (COOH) modified BMB, 137.17/0.0070 = 19,595.7.
For Amino-Spacer² - 2 C (COOH) modified BMB, 411.52/0.063 = 6,532.1.
For Amino-Spacer¹ - 1 C (COOH) modified BMB, 137.17/0.19 = 721.95.
From the above table, Amino-Spacer³ - 3 C (COOH) modified BMB showed larger dynamic range than Amino-Spacer¹ - 1 C (COOH) or Amino-Spacer² - 2 C (COOH) modified BMB.
The Amino-Spacer³ - 3 C (COOH) modified BMB showed higher capacity (higher MFI) than Amino-Spacer¹ - 1 C (COOH) or Amino-Spacer² - 2 C (COOH) modified BMB.
The Amino-Spacer³ - 3 C (COOH) modified BMB showed higher sensitivity than Amino-Spacer¹ - 1 C (COOH) or Amino-Spacer² - 2 C (COOH) modified BMB.

### Example 5

Various Amino-Spacerⁿ-n C (COOH) modified SU-8 BMB and Hybridization with 3-fold serial diluted GAPDH Target with 50-mer capture probe, 25 min at 52° C., 820 rpm, SAPE 5 μg/mL, 12.5 min. A summary of the MFI observed for Compound 1, Compound 2, Compound 3, Compound 4, Compound 5, and Compound 6 with varying concentrations of GAPDH Target is presented in Table 9.

TABLE 9

| | Compound 1 | Compound 2 | Compound 3 | Compound 4 | Compound 5 | Compound 6 | |
|---|---|---|---|---|---|---|---|
| | (Amino- | (Amino- | (Amino- | (Amino- | (Amino- | (Amino- | |
| GAPDH | Spacer¹ -1 | Spacer² -2 | Spacer³ -3 | Spacer⁴ -4 | Spacer⁵ -5 | Spacer⁶ -6 | |
| Target | C) | C) | C) | C) | C) | C) | |
| fmol/μL | MFI | MFI | MFI | MFI | MF | MFI | Blank |
| 0.00 | 2 | 34 | 2 | 70 | 141 | 59 | 2 |
| 0.00026 | 1 | 54 | 1 | 127 | 95 | 70 | 1 |
| 0.00077 | 3 | 34 | 3 | 97 | 47 | 36 | 3 |
| 0.0023 | 5 | 73 | 5 | 98 | 4 | 106 | 5 |
| 0.0070 | 9 | 145 | 5 | 227 | 120 | 190 | 7 |
| 0.0021 | 59 | 288 | 188 | 385 | 345 | 245 | 5 |
| 0.063 | 211 | 695 | 644 | 918 | 552 | 745 | 5 |
| 0.19 | 624 | 1850 | 1971 | 2497 | 1619 | 1954 | 4 |
| 0.56 | 1725 | 4820 | 5427 | 6477 | 3474 | 5345 | 1 |
| 1.69 | 4997 | 12690 | 14186 | 15803 | 7541 | 13254 | 5 |
| 5.08 | 10538 | 24942 | 27999 | 28709 | 18676 | 26592 | 3 |
| 15.24 | 22565 | 37609 | 43590 | 41632 | 30997 | 41900 | 5 |
| 45.72 | 34729 | 56380 | 80620 | 81640 | 111784 | 79315 | 7 |
| 137.17 | 41468 | 74025 | 157754 | 130051 | 188426 | 149733 | 6 |
| 411.52 | 46737 | 80343 | 188054 | 173706 | 188054 | 188054 | 2 |
| 1235 | 47622 | 89602 | 188524 | 188355 | 188524 | 188524 | 2 |
| 3704 | 46972 | 96772 | 186995 | 186995 | 186995 | 186995 | 1 |
| 11111 | 50755 | 90452 | 185734 | 185734 | 185734 | 185734 | 5 |

Amino-Spacer³ - 3 C (COOH) modified BMB showed very low background and higher capacity than Amino-Spacer¹ - 1 C (COOH) and Amino-Spacer² - 2 C (COOH) modified BMB. Amino-Spacer⁴ - 4 C (COOH), Amino-Spacer⁵ - 5 C (COOH), and Amino-Spacer⁶ - 6 C (COOH) modified BMB showed higher saturation capacity and higher background compared to Amino-Spacer³ - 3 C (COOH) modified BMB.

The specification will be further understood in view of the following non-limiting embodiments.

Embodiment 1.A biological assay device comprising a reactive solid substrate linked to one or more amino-spacerⁿ-n Carboxyl (COOH) moieties, said amino-spacerⁿ-n Carboxyl (COOH) moieties having a first end comprising a terminal amino group linked to the solid substrate, and a second end comprising n terminal carboxylic acid functionalities linked to one or more biological probe molecules;
wherein each of the one or more amino-spacerⁿ-n Carboxyl (COOH) moieties comprises two or more branches each independently comprising one or more nitrogen atoms and a chain comprising a linear C₁₋₂₀ alkylene, C₁₋₆ alkylene oxide, or polyalkylene oxide; and
n is an integer from 2 to 6.

Embodiment 2. The biological assay device of embodiment 1, wherein the one or more amino-spacerⁿ-n Carboxyl (COOH) moieties comprise two or more branches derived from combinations of initial amino-spacerⁱ-i C (COOH), epoxy-spacer^{m}-m C (COOH), bifunctional epoxy compounds, and multifunctional amines, wherein
i and m are each independently integers from 1 to 20.

Embodiment 3. The biological assay device of embodiment 1, wherein the one or more amino-spacerⁿ-n Carboxyl (COOH) moieties have a structure selected from amino-spacer²-2 C (COOH), amino-spacer³-3 C (COOH), amino-spacer^{4-I}-4 C (COOH) [1 C+3 C], amino-spacer^{4-I}-4 C (COOH) [2 C+2 C], amino-spacer^{5-I}-5 C (COOH) [2 C+3 C], amino-spacer^{5-I}-5 C (COOH) [1 C+4 C (1 C+3 C)], amino-spacer^{5-I}-5 C (COOH) [1 C+4 C (2 C+2 C)], and amino-spacer^{6-(c)}-6 C (COOH) [3 C+3 C]:

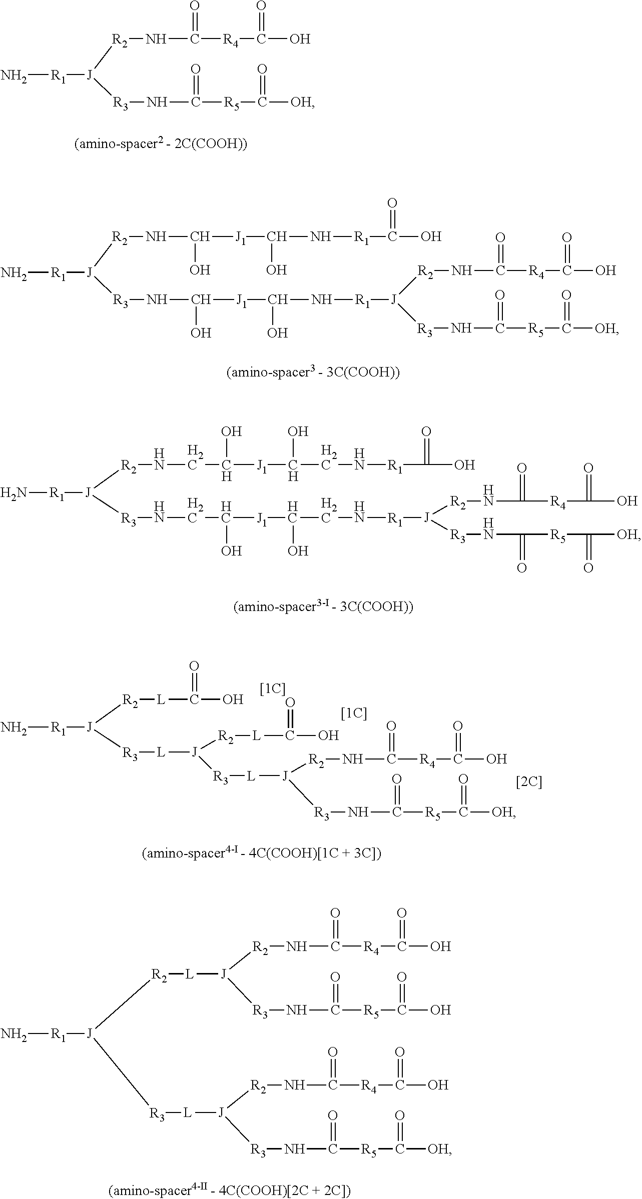

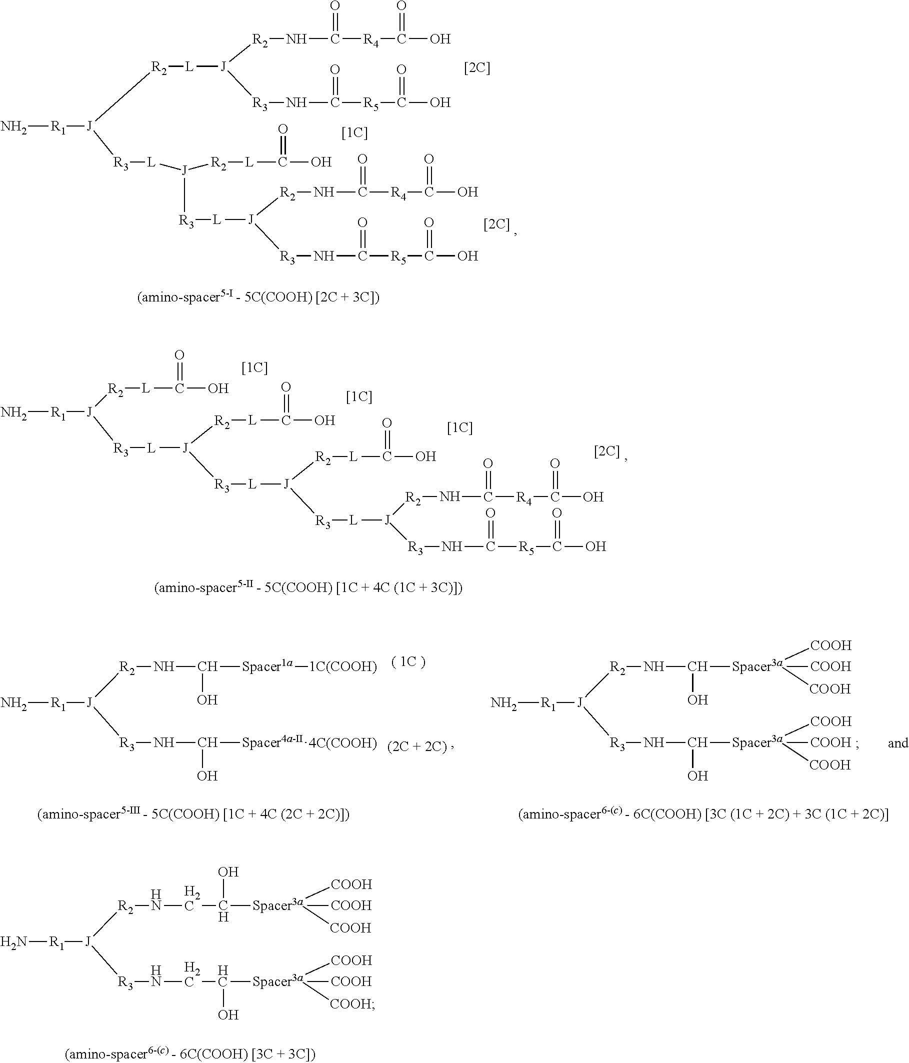

wherein
each R₁, R₂, and R₃ is independently C₁₋₂₀ alkylene, C₁₋₆ alkylene oxide, or polyalkylene oxide;
each R₄ and R₅ is independently C₁₋₂₀ alkylene;
each J is N, or a straight chain or branched C₁₋₆ alkylene comprising a quaternary carbon center;
each J₁ is independently C₁₋₂₀ alkylene, C₁₋₆ alkylene oxide, or polyalkylene oxide;

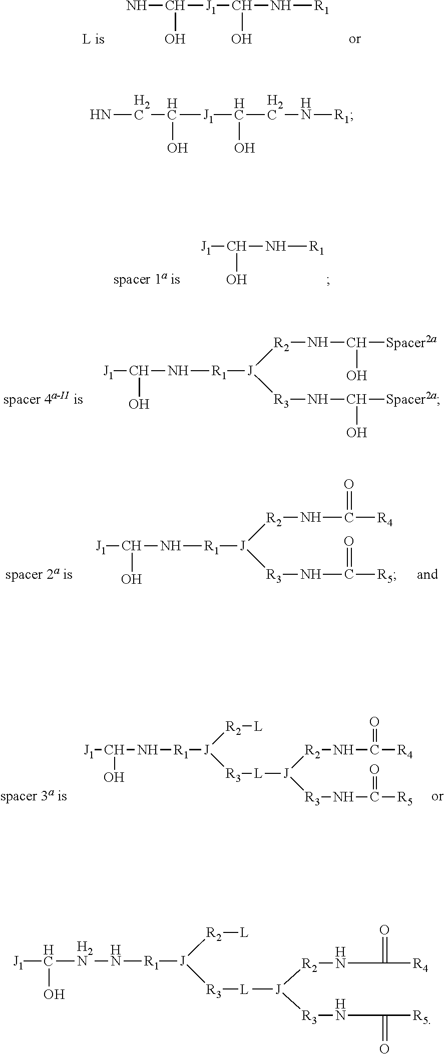

Embodiment 4. The biological assay device of embodiment 3, wherein at least one J is CH.

Embodiment 5. The biological assay device of embodiment 4, wherein each J is CH.

Embodiment 6. The biological assay device of embodiment 3, wherein at least one J is N.

Embodiment 7. The biological assay device of embodiment 6, wherein each J is N.

Embodiment 8. The biological assay device of embodiment 3, wherein at least one J₁ is C₁₋₂₀ alkylene.

Embodiment 9. The biological assay device of embodiment 8, wherein each J₁ is C₁₋₂₀ alkylene.

Embodiment 10. The biological assay device of embodiment 3, wherein at least one J₁ is C₁₋₆ alkylene oxide.

Embodiment 11. The biological assay device of embodiment 10, wherein each J₁ is C₁₋₆ alkylene oxide.

Embodiment 12. The biological assay device of embodiment 3, wherein at least one J₁ is polyalkylene oxide.

Embodiment 13. The biological assay device of embodiment 12, wherein each J₁ is polyalkylene oxide.

Embodiment 14. The biological assay device of embodiment 3, wherein at least one R₁ is C₁₋₂₀ alkylene.

Embodiment 15. The biological assay device of embodiment 14, wherein each R₁ is C₁₋₂₀ alkylene.

Embodiment 16. The biological assay device of embodiment 3, wherein at least one R₁ is C₁₋₆ alkylene oxide.

Embodiment 17. The biological assay device of embodiment 16, wherein each R₁ is C₁₋₆ alkylene oxide.

Embodiment 18. The biological assay device of embodiment 3, wherein at least one R₁ is polyalkylene oxide.

Embodiment 19. The biological assay device of embodiment 18, wherein each R₁ is polyalkylene oxide.

Embodiment 20. The biological assay device of embodiment 3, wherein at least one R₂ is C₁₋₂₀ alkylene.

Embodiment 21. The biological assay device of embodiment 20, wherein each R₂ is C₁₋₂₀ alkylene.

Embodiment 22. The biological assay device of claim 3, wherein at least one R₂ is C₁₋₆ alkylene oxide.

Embodiment 23. The biological assay device of embodiment 22, wherein each R₂ is C₁₋₆ alkylene oxide.

Embodiment 24. The biological assay device of embodiment 3, wherein at least one R₂ is polyalkylene oxide.

Embodiment 25. The biological assay device of embodiment 24, wherein each R₂ is polyalkylene oxide.

Embodiment 26. The biological assay device of embodiment 3, wherein at least one R₃ is C₁₋₂₀ alkylene.

Embodiment 27. The biological assay device of embodiment 26, wherein each R₃ is C₁₋₂₀ alkylene.

Embodiment 28. The biological assay device of embodiment 3, wherein at least one R₃ is C₁₋₆ alkylene oxide.

Embodiment 29. The biological assay device of embodiment 28, wherein each R₃ is C₁₋₆ alkylene oxide.

Embodiment 30. The biological assay device of embodiment 3, wherein at least one R₃ is polyalkylene oxide.

Embodiment 31. The biological assay device of embodiment 30, wherein each R₃ is polyalkylene oxide.

Embodiment 32. The biological assay device of embodiment 3, wherein C₁₋₆ alkylene oxide is ethylene oxide or propylene oxide.

Embodiment 33. The biological assay device of embodiment 32, wherein polyalkylene oxide is polyethylene oxide (PEO) or polypropylene oxide (PPO).

Embodiment 34. The biological assay device of embodiment 33, wherein the polyethylene oxide or polypropylene oxide has an average molecular weight of 200 g/mol or greater.

Embodiment 35. The biological assay device of embodiment 34, wherein the polyethylene oxide or polypropylene oxide has an average molecular weight of 200 g/mol to 20,000 g/mol.

Embodiment 36. The biological assay device of embodiment 1, wherein the solid substrate comprises one or more reactive functionalities selected from epoxy, amino, carboxyl, and thiol groups.

Embodiment 37. The biological assay device of embodiment 36, wherein the epoxy groups comprise ethylene glycol diglycidyl ether, poly (ethylene glycol) diglycidyl ether, or poly (propylene glycol) diglycidyl ether.

Embodiment 38. The biological assay device of embodiment 1, wherein the solid substrate is selected from the group consisting of films, microporous membranes, beads, magnetic beads, barcoded magnetic beads, and nickel barcoded magnetic beads.

Embodiment 39. The biological assay device of embodiment 38, wherein the barcoded magnetic beads comprise a series of alternating light transmissive and opaque sections, with relative positions, widths and spacing resembling a 1D or 2D bar code image.

Embodiment 40. The biological assay device of embodiment 39, wherein the opaque sections comprise a paramagnetic material.

Embodiment 41. The biological assay device of embodiment 40, wherein the paramagnetic material is Ni.

Embodiment 42. The biological assay device of embodiment 1, wherein the one or more biological probe molecules are selected from the group consisting of antibodies, proteins, oligopeptides, peptide sequences, and oligonucleosides, DNA, and RNA.

Embodiment 43. The biological assay device of embodiment 1, wherein the two or more branches comprising one or more nitrogen atoms comprise a multifunctional amine-derived moiety.

Embodiment 44. The biological assay device of embodiment 43, wherein the multifunctional amine-derived moiety is a moiety derived from amino dextran, polyethylenimine, poly (vinyl amine), poly (allyl amine), poly (ethylene glycol), or poly (propylene glycol) based trifunctional amines.

Embodiment 45. The biological assay device of embodiment 1, wherein the reactive solid substrate comprises an epoxy-containing SU-8 barcoded magnetic bead.

Embodiment 46. The biological assay device of embodiment 45, wherein the biological assay device shows higher fluorescence intensity (MFI) after coupling with fluorescent biological probes than biological assay devices that do not comprise one or more amino-spacerⁿ-n Carboxyl (COOH) moieties.

Embodiment 47. The biological assay device of embodiment 46, wherein the biological assay device shows higher fluorescence intensity (MFI) and lower background fluorescence after coupling with fluorescent biological probes than biological assay devices that do not comprise one or more amino-spacerⁿ-n Carboxyl (COOH) moieties.

Embodiment 48. The biological assay device of embodiment 47, wherein the biological assay device shows a higher dynamic range after coupling with biological probes than biological assay devices that do not comprise one or more amino-spacerⁿ-n Carboxyl (COOH) moieties.

Embodiment 49. A method of detecting a substance in a biological sample, comprising contacting the biological sample with the biological assay device of claim 1.

Embodiment 50. The method of embodiment 49, wherein the substance is selected from an antigen, antibody, virus, pathogen, DNA, and RNA.

Embodiment 51. A method of preparing an amino-spacerⁿ-n Carboxyl (COOH) moiety, the method comprising admixing one or more initial amino-spacerⁱ-i C (COOH) moieties, one or more epoxy-spacer^{m}-m C (COOH) moieties, one or more multifunctional amines, and/or one or more diglycidyl ethers of C₁₋₆ alkylene oxides or polyalkylene oxides,
wherein n is an integer from 2 to 6, and
i and m are each independently integers from 1 to 20.

Embodiment 52. The method of embodiment 51, wherein the one or more multifunctional amines comprise one or more trifunctional amines.

What is claimed is:

1. A biological assay device comprising a reactive solid substrate linked to one or more amino-spacerⁿ-n Carboxyl (COOH) moieties, said amino-spacerⁿ-n Carboxyl (COOH) moieties having a first end comprising a terminal amino group linked to the solid substrate, and a second end comprising n terminal carboxylic acid functionalities linked to one or more biological probe molecules;
wherein each of the one or more amino-spacerⁿ-n Carboxyl (COOH) moieties comprises two or more branches each independently comprising one or more nitrogen atoms and a chain comprising a linear C₁₋₂₀ alkylene, C₁₋₆ alkylene oxide, or polyalkylene oxide; and
n is an integer from 2 to 6.

2. The biological assay device of claim 1, wherein the one or more amino-spacerⁿ-n Carboxyl (COOH) moieties comprise two or more branches derived from combinations of initial amino-spacerⁱ-i C (COOH), epoxy-spacer^{m}-m C (COOH), bifunctional epoxy compounds, and multifunctional amines, wherein i and m are each independently integers from 1 to 20.

3. The biological assay device of claim 1, wherein the one or more amino-spacerⁿ-n Carboxyl (COOH) moieties have a structure selected from amino-spacer²⁻² C (COOH), amino-spacer³-3 C (COOH), amino-spacer^{4-I}-4 C (COOH) [1 C+3 C], amino-spacer^{4-II}-4 C (COOH) [2 C+2 C], amino-spacer^{5-I}-5 C (COOH) [2 C+3 C], amino-spacer^{5-II}-5 C (COOH) [1 C+4 C (1 C+3 C)], amino-spacer^{5-III}-5 C (COOH) [1 C+4 C (2 C+2 C)], and amino-spacer^{6-(c)}-6 C (COOH) [3 C+3 C]: 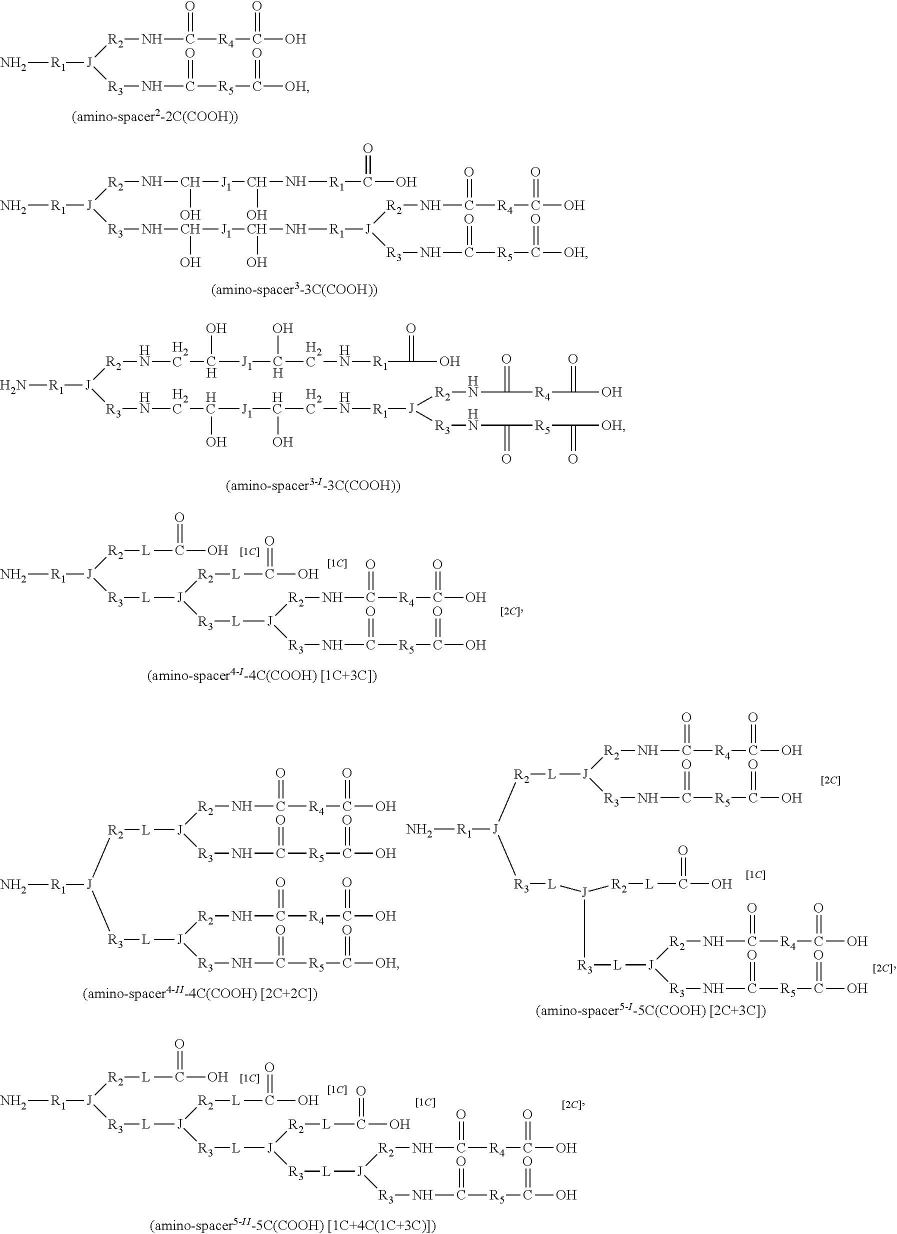 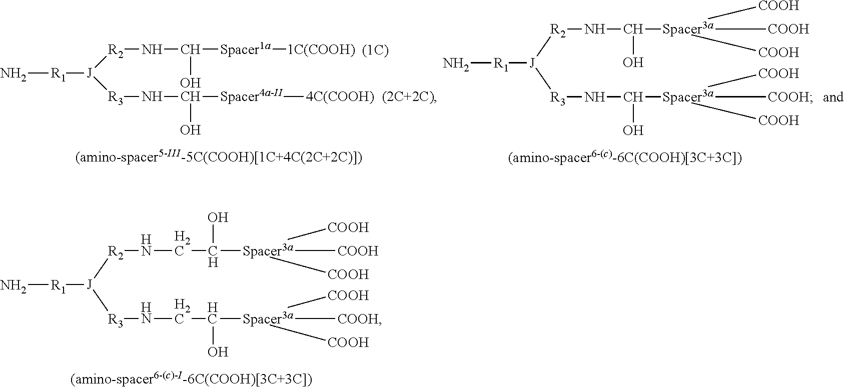
wherein
each R₁, R₂, and R₃ is independently C₁₋₂₀ alkylene, C₁₋₆ alkylene oxide, or polyalkylene oxide;
each R₄ and R₅ is independently C₁₋₂₀ alkylene;
each J is N, or a straight chain or branched C₁₋₆ alkylene comprising a quaternary carbon center;
each J₁ is independently C₁₋₂₀ alkylene, C₁₋₆ alkylene oxide, or polyalkylene oxide;

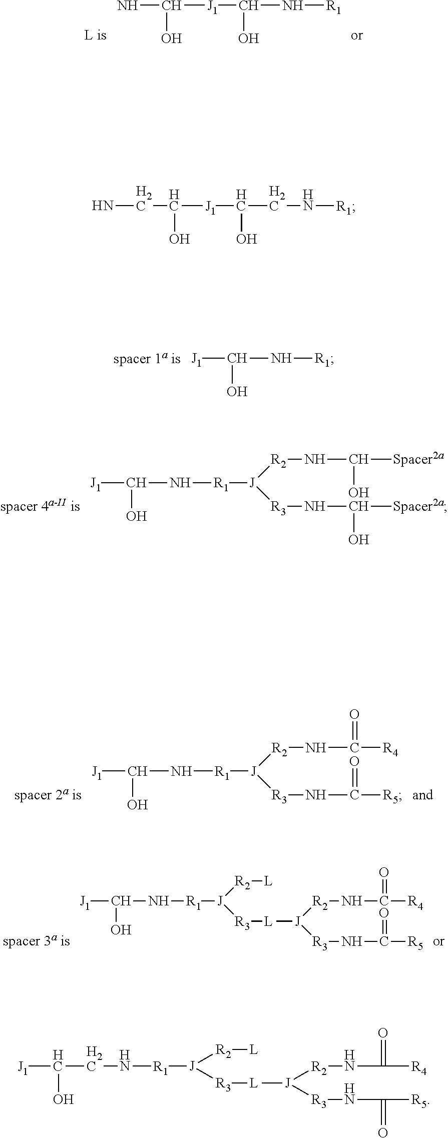

4. The biological assay device of claim 2, wherein the bifunctional epoxy compounds are poly (ethylene glycol) diglycidyl ether or poly (propylene glycol) diglycidyl ether.

5. The biological assay device of claim 4, wherein the poly (ethylene glycol) diglycidyl ether or poly (propylene glycol) diglycidyl ether has an average molecular weight of 200 g/mol to 20,000 g/mol.

6. The biological assay device of claim 2, wherein the bifunctional epoxy compounds comprise ethylene glycol diglycidyl ether, 1,4-Butanediol diglycidyl ether, 1,6-Hexanediol diglycidyl ether, etc.

7. The biological assay device of claim 1, wherein the solid substrate comprises one or more reactive functionalities selected from epoxy, amino, carboxyl, and thiol groups.

8. The biological assay device of claim 1 wherein the solid substrate is selected from the group consisting of films, microporous membranes, beads, magnetic beads, barcoded magnetic beads, and nickel barcoded magnetic beads.

9. The biological assay device of claim 8 wherein the barcoded magnetic beads comprise a series of alternating light transmissive and opaque sections, with relative positions, widths and spacing resembling a 1D or 2D bar code image.

10. The biological assay device of claim 9 wherein the opaque sections comprise a paramagnetic material.

11. The biological assay device of claim 10, wherein the paramagnetic material is Ni.

12. The biological assay device of claim 1, wherein the one or more biological probe molecules are selected from the group consisting of antibodies, proteins, oligopeptides, peptide sequences, and oligonucleosides, DNA, and RNA.

13. The biological assay device of claim 1, wherein the two or more branches comprising one or more nitrogen atoms comprise a multifunctional amine-derived moiety.

14. The biological assay device of claim 13, wherein the multifunctional amine-derived moiety is a moiety derived from amino dextran, Tris (aminoalkyl) amine, polyethylenimine, poly (vinyl amine), poly (allyl amine), poly (ethylene glycol) or poly (propylene glycol) based trifunctional amines.

15. The biological assay device of claim 1, wherein the reactive solid substrate comprises an epoxy-containing SU-8 barcoded magnetic bead.

16. The biological assay device of claim 1, wherein the biological assay device shows higher fluorescence intensity (MFI) and lower background fluorescence after coupling with fluorescent biological probes than biological assay devices that do not comprise one or more amino-spacerⁿ-n Carboxyl (COOH) moieties.

17. The biological assay device of claim 1, wherein the biological assay device shows a higher dynamic range after coupling with biological probes than biological assay devices that do not comprise one or more amino-spacerⁿ-n Carboxyl (COOH) moieties.

18. A method of detecting a substance in a biological sample, comprising contacting the biological sample with the biological assay device of claim 1.

19. The method of claim 18, wherein the substance is selected from an antigen, antibody, virus, pathogen, DNA, and RNA.

20. A method of preparing an amino-spacerⁿ-n Carboxyl (COOH) moiety, the method comprising admixing one or more initial amino-spacerⁱ-i C (COOH) moieties, one or more epoxy-spacer^{m}-m C (COOH) moieties, one or more multifunctional amines, and/or one or more Ethlene glycol diglycidyl ethers, 1,4-Butanediol diglycidyl ether, 1,6-Hexanediol diglycidyl ether, poly (ethylene glycol) diglycidyl ether or Poly (propylene glycol) diglycidyl ether with average molecular weight of 200 g/mol or greater, wherein n is an integer from 2 to 6, and i and m are each independently integers from 1 to 20.
